(19) United States
(12) Patent Application Publication
(10) Pub. No.: US 2017/0328922 A1
(43) Pub. Date: Nov. 16, 2017

(54) METHODS AND PRODUCTS FOR LABELLING LIPIDS

(71) Applicants: UNIVERSITY OF SOUTH AUSTRALIA, Adelaide, South Australia (AU); CURTIN UNIVERSITY OF TECHNOLOGY, Perth, Western Australia (AU)

(72) Inventors: Doug Brooks, Cheltenham (AU); Sally Plush, Mile End (AU); Massimiliano Massi, Wilson (AU)

(73) Assignees: UNIVERSITY OF SOUTH AUSTRALIA, Adelaide, South Australia (AU); CURTIN UNIVERSITY OF TECHNOLOGY, Perth, Western Australia (AU)

(21) Appl. No.: 15/528,166

(22) PCT Filed: Mar. 19, 2015
(86) PCT No.: PCT/AU2015/000159

§ 371 Date: May 19, 2017

(30) Foreign Application Priority Data
Nov. 21, 2014 (AU) 2014904715

(51) Int. Cl. G01N 33/92; C07D 471/04; G01N 33/50; G01N 33/58; C07D 257/04; G01N 33/574; C07F 13/00

(52) U.S. Cl. CPC G01N 33/92; G01N 33/574; C07D 471/04; C07F 13/00; G01N 33/586; C07D 257/04; G01N 33/5005; G01N 2405/02; G01N 2405/06; G01N 2405/10

(57) ABSTRACT

The present disclosure relates to methods and products for labelling, binding and/or detection of lipids. Certain embodiments provide a method of labelling a lipid. The method comprises exposing the lipid to a complex comprising a transition metal carbonyl compound, a conjugated bidentate ligand and a tetrazolato compound, and thereby labelling the lipid by binding the complex to the lipid.

### PRIORITY CLAIM

This application claims priority to Australian provisional patent application number 2014904715 filed on 21 Nov. 2014, the content of which is hereby incorporated by reference.

### FIELD

The present disclosure relates to methods and products for labelling, binding and/or detection of lipids.

### BACKGROUND

Molecular probes are important tools, including for research purposes and for medical diagnostic purposes. A variety of probes are available for many different types of biological molecular species, such as DNA, RNA and a variety of proteins. However, the chemical nature of some molecular species makes them a difficult target for the design and use of probes. The absence of probes to certain types of molecular species is a major limitation in the investigation of many biological processes that involve such molecular species.

Cellular lipids, in particular, are difficult to visualise with probes and most of the currently available lipid probes have a variety of limitations, such as requiring fixation of the cells, which can impair ultrastructure and also prevent live cell imaging. For example, probes such as Oil-Red-O and Nile Red both require sample fixation and both have issues with solubility. Lipid fixation is particularly problematic for alcohol based solvents and fixatives, which cause fusion of lipid droplets and or extraction of lipids from cell/tissue samples.

The chemical properties of lipids also make the design of specific probes to different types of lipid very difficult. As such, most of the lipid probes identified to date have little or no specificity for different types of lipids.

A further deficiency of many probes is that they are not suitable for imaging of live cells. This is particularly difficult in the case of lipids. The ability to translate live cell imaging into practice not only has important implications for visualizing normal cell function, but also has direct significance for the investigation of many diseases. As such, the development of probes that are suitable for live cell imaging has become an important area for development, not least for the reason that such probes are likely to provide diagnostic and prognostic tools that can be applied to discern specific patient pathologies.

A lack of appropriate tools for the investigation of lipid biology has in particular hindered progress in this area, particularly for example for live cell imaging. Altered lipid biology has been linked to a variety of important diseases and the lack of suitable probes remains a significant limitation.

Accordingly, for a variety of reasons there is a need for improved probes that can label or detect lipids.

### SUMMARY

The present disclosure relates to methods and products for the labelling, binding and/or detection of lipids.

Certain embodiments of the present disclosure provide a method of labelling a lipid, the method comprising exposing the lipid to a complex comprising a transition metal carbonyl compound, a conjugated bidentate ligand and a tetrazolato compound, and thereby labelling the lipid by binding the complex to the lipid.

Certain embodiments of the present disclosure provide a method of labelling one or more of an endosome, a lysosome, an autophagosome and a lipid droplet, the method comprising exposing one or more of an endosome, a lysosome, an autophagosome, and a lipid droplet to a complex comprising a transition metal carbonyl compound, a conjugated bidentate ligand and a tetrazolato compound and thereby labelling one or more of the endosome, the lysosome, the autophagosome and the lipid droplet by binding of the complex to one or more of the endosome, the lysosome, the autophagosome and the lipid droplet.

Certain embodiments of the present disclosure provide a method of detecting a lipid, the method comprising binding to the lipid a complex comprising a transition metal carbonyl compound, a conjugated bidentate ligand and a tetrazolato compound and detecting the complex bound to the lipid.

Certain embodiments of the present disclosure provide a method of detecting one or more of an endosome, a lysosome, an autophagosome and a lipid droplet, the method comprising binding to one or more of an endosome, a lysosome, an autophagosome and a lipid droplet a complex comprising a transition metal carbonyl compound, a conjugated bidentate ligand and a tetrazolato compound and thereby detecting one or more of the endosome, the lysosome, the autophagosome and the lipid droplet by detecting the complex bound to the endosome and/or the lysosome.

Certain embodiments of the present disclosure provide a method of intracellular imaging of a cell, the method comprising exposing a cell to a complex comprising a transition metal carbonyl compound, a conjugated bidentate ligand and a tetrazolato compound and intracellularly imaging lipids in the cell bound to the complex.

Certain embodiments of the present disclosure provide an intracellular imaging agent, the agent comprising a complex comprising a transition metal carbonyl compound, a conjugated bidentate ligand and a tetrazolato compound.

Certain embodiments of the present disclosure provide a method of intracellularly imaging a cell, the method comprising exposing a cell to an agent as described herein and intracellularly imaging lipids in the cell bound to the agent.

Certain embodiments of the present disclosure provide a kit for intracellular imaging of cells, the kit comprising a complex comprising a transition metal carbonyl compound, a conjugated bidentate ligand and a tetrazolato compound.

Certain embodiments of the present disclosure provide a kit for labelling lipids, the kit comprising a complex comprising a transition metal carbonyl compound, a conjugated bidentate ligand and a tetrazolato compound.

Certain embodiments of the present disclosure provide a kit for labelling intracellular structures containing lipid, the kit comprising a complex comprising a transition metal carbonyl compound, a conjugated bidentate ligand and a tetrazolato compound.

Certain embodiments of the present disclosure provide a method of detecting a disease, condition or state in a subject associated with altered or dysfunctional endosomal functionality, lysosomal functionality and/or autophagy, the method comprising labelling one or more of endosomes, lysosome, autophagosomes and lipid droplets from the subject with a complex comprising a transition metal carbonyl compound, a conjugated bidentate ligand and a tetrazolato compound and detecting the disease, condition or state on the basis of the one or more endosomes, lysosomes autophagosomes and lipid droplets so labelled.

Certain embodiments of the present disclosure provide a method of detecting a disease, condition or state in a subject associated with altered or dysfunctional endosomal functionality, lysosomal functionality and/or autophagy, the method comprising exposing one or more cells from the subject to a complex comprising a transition metal carbonyl compound, a conjugated bidentate ligand and a tetrazolato compound to label the one or more cells and detecting the disease, condition or state on the basis of the cells so labelled.

A method of detecting a disease, condition or state in a subject associated with altered or dysfunctional lipid intake, metabolism, processing, biogenesis or accumulation, the method comprising exposing one or more cells from the subject to a complex comprising a transition metal carbonyl compound, a conjugated bidentate ligand and a tetrazolato compound to label the one or more cells and detecting the disease, condition or state on the basis of the cells so labelled.

Certain embodiments of the present disclosure provide a method of identifying a compound for labelling a lipid, the method comprising:
providing a candidate complex comprising a transition metal carbonyl compound, a conjugated bidentate ligand and a tetrazolato compound;
determining the ability of the candidate complex to bind to a lipid; and
identifying the candidate complex as a compound for labelling a lipid on the basis of the complex binding to the lipid.

Certain embodiments of the present disclosure provide a method of identifying a compound for intracellular imaging of a cell, the method comprising:
providing a candidate complex comprising a transition metal carbonyl compound, a conjugated bidentate ligand and a tetrazolato compound;
determining the ability of the candidate complex to bind to a lipid in the cell; and
identifying the candidate complex as a compound for labelling a lipid on the basis of the compound binding to the lipid in the cell.

Certain embodiments of the present disclosure provide a method of determining intracellular pH of a cell, the method comprising:
exposing the cell to a complex comprising a transition metal carbonyl compound, a conjugated bidentate ligand and a tetrazolato compound;
determining one or more emission characteristics from the complex in the cell; and
determining the intracellular pH on the basis of the one or more emission characteristics determined.

Certain embodiments of the present disclosure provide a method of identifying a cancerous cell, the method comprising exposing a cell to a complex comprising a transition metal carbonyl compound, a conjugated bidentate ligand and a tetrazolato compound and identifying the cell as a cancerous cell by increased labelling of the cell by the complex.

Certain embodiments of the present disclosure provide a method of identifying a cancerous cell in a subject, the method comprising exposing one or more cells from the subject to a complex comprising a transition metal carbonyl compound, a conjugated bidentate ligand and a tetrazolato compound to label the one or more cells and identifying a cancerous cell by increased labelling of the one or more cells by the complex.

Other embodiments are disclosed herein.

### BRIEF DESCRIPTION OF THE FIGURES

Certain embodiments are illustrated by the following figures. It is to be understood that the following description is for the purpose of describing particular embodiments only and is not intended to be limiting with respect to the description.

FIG. 1 shows that Rhenium probe is detected in Drosophila fat body lipid droplets. Confocal micrographs of fat body cells explanted from WT¹¹¹⁸ Drosophila larvae and pupae at either −4 h PF (A-A^{II}), 0 h PF (B-B^{II}), or +2 h PF (C-C^{II}) and stained with either Rhenium probe (grey scale in A, B, C and red in A^{II}, B^{II}, C^{II};) or LysoTracker® green (grey scale in A^{I}, B^{I}, C^{I} Green in A^{II}, B^{II}, C^{II}). Arrows indicate colocation of Rhenium probe to LysoTracker® green positive compartments, while arrowheads indicated lipid droplets. Scale bars=10 μm.

FIG. 2 shows that Rhenium probe colocates with Atg8aGFP upon starvation and during pupal development. Confocal micrographs of Drosophila fat body tissue from larvae at −4 h PF (A-D^{II}) or pupae at +2 h PF (E-E^{II}), either stained with Rhenium probe (grey scale in A-E or red in A^{II}-E^{II};) or expressing Atg8a-GFP (grey scale in AI-EI green in A^{II}-E^{II}). Images from larvae at −4 h PF on standard feeding medium (A-A^{II}), −4 h PF larvae deprived of amino acids for 4 h (B-B^{II}), −4 h PF larvae deprived of sugar for 4 h (C-C^{II}), −4 h PF larvae starved for 4 h (deprived of amino acids and sugar; D-D^{II}) and pupae at +2 h PF (E-E^{II}). Arrowheads indicate Rhenium probe collocating with Atg8aGFP positive compartments. Scale bars=10 μm.

FIG. 3 shows neutral lipid staining with Oil Red O and Atg8aGFP phagosomes during either larval starvation or pupal development. Confocal micrographs of fixed fat body tissue from Drosophila larvae at −4 h PF (A-D^{II}) or pupae at +2 h PF (E-E^{II}), and either stained with Oil Red O (grey scale in A-E or red in A^{II}-E^{II}) or expressing Atg8aGFP (grey scale in A^{I}-E^{I} or green in A^{II}-E^{II}). Images from larvae at −4 h PF on standard feeding medium (A-A^{II}), −4 h PF larvae deprived of amino acids for 4 h (B-B^{II}), −4 h PF larvae deprived of sugar for 4 h (C-C^{II}), −4 h PF larvae starved for 4 h (D-D^{II}) or pupae at +2 h PF (E-E^{II}). Scale bars=10 μm.

FIG. 4 shows that constitutive activation of autophagy and Atg9RNAi alter Rhenium probe distribution. Confocal micrographs of Drosophila fat body tissue from larvae at −8 h PF (A-B^{II}), −4 h PF (C-H^{II}) or pupae at +2 h PF (I-J^{II}), incubated either with Rhenium probe (grey scale in A-I, or B-J; or red in A^{II-III} and B^{II}-J^{II}) or with LysoTracker® green (Grey scale in A^{I-II} or B^{I}-J^{I}; or green in A^{II-III} and B^{II-III}). Fat bodies from controls (A-I^{II}), TorTED (B-B^{II}) or Atg9RNAi (D-J^{II}) were either larvae fed on standard feeding medium (A-D^{II}), larvae deprived of amino acids for 4 h (E-F^{II}), larvae starved (-amino acids, -sugar) for 4 h (G-H^{II}) or pupae fed on standard feeding medium (I-J^{II}). Arrows indicate Rhenium probe localised to LysoTracker® green positive compartments and arrowheads indicate lipid droplets. Scale bars=10 μm.

FIG. 5 shows quantification of Rhenium probe and LysoTracker™ positive compartments during starvation and development. Histogram showing the number of Rhenium probe and LysoTracker™ positive compartments, present in fat body tissues. Tor^{TED} autophagy induced by Tor inactivation; Atg9^{RNAi} depletion to impair autophagy. Results are presented as the mean±SEM.

FIG. 6 shows that the Rhenium molecular probe PhenCyano interacts with lipids and detergents. Reactivity of Rhenium probe with compounds spotted onto PDF membrane, incubated either with or without (negative control) Rhenium probe and detected at 575-605 nm.

FIG. 7 shows that the Rhenium molecular probes PhenCyano and PhenPyridyl interact with specific lipids.

FIG. 8 shows that the Rhenium molecular probes PhenEster, BiphenCyano and BiphenEster interact with specific lipids.

FIG. 9 shows absorption and emission profiles of complexes 1 and 2 from a diluted (ca. 10⁻⁵ M) air-equilibrated H₂O/DMSO 99:1 solution at room temperature.

FIG. 10 shows microscopy images of PhenCyano and PhenPyridyl probe incubated with live samples, as indicated in image. Left confocal images, right fluorescence microscopy images.

FIG. 11 shows paraffin embed brain sections from control and lysosome storage disorder, MPSIIA mice, stained with rhenium PhenCyano molecular probe.

FIG. 12 shows that the rhenium molecular probe PhenCyano localised with Atg8aGFP upon starvation and during pupal development. Confocal micrographs of Drosophila fat body tissue from larvae at −4 h PF (A-DII) or pupae at +2 h PF (E-EII), stained with Rhenium molecular probe (grey scale in A, B, C, D and E; red in AII, BII, CII, DII and EII;) and expressing Atg8a-GFP (grey scale in AI, BI, CI, DI and EI; green in AII, BII, CII, DII and EII). Tissue from larvae at −4 h PF on standard feeding medium (A-AII), −4 h PF larvae deprived of amino acids for 4 h (B-BII), −4 h PF larvae deprived of sugar for 4 h (C-CII), −4 h PF larvae starved for 4 h (deprived of amino acids and sugar; D-DII) and pupae at +2 h PF (E-EII). Arrowheads indicate Rhenium molecular probe collocating with Atg8aGFP positive compartments (C-EII), arrows indicate Rhenium molecular probe alone (C-EII). Scale bars=10 μm.

FIG. 13 shows spectral profiling of the Rhenium molecular probe PhenCyano in acidic compartments and lipid droplets. (A) Confocal micrographs of Drosophila fat body at +2 h PF stained with Rhenium molecular probe showing spectral profile of probe in acidic compartments (cyan) and lipid droplets (green/yellow). (B) Spectral emissions profile of Rhenium molecular probe in different subcellular compartments (acidic compartments=solid line: lipid droplets=dashed line) relative to emission maxima of acidic compartment (intensity at λem 480 nm).

FIG. 14 shows the pH profile of the PhenCyano probe as expressed as a function of the ratio between the emission intensity of complex 1 at 440 m normalized against 560 nm. The x-axis shows the pH and the y-axis the ratio of the intensity at the two wavelengths.

FIG. 15 shows the labelling of the PhenCyano (“Cyano”) probe and the PhenPyridyl (“Lyso”) probe in prostate cells. The cells labelled PNT2 and PNT1a are non-malignant and the 22RV1 and LNCaP cells are malignant. Panel A shows comparison of PNT2 and 22RV1 cells with the probes. Panel B shows comparison of PNT1a and LNCaP cells with the probes. In this figure, the imaging was adjusted so that all were captured at the same level with identical settings.

FIG. 16 shows the same image as provided in FIG. 15 Panel A, at different levels. FIG. 16A shows imaging captured at the same level with identical settings. FIG. 16B show imaging at an optimal level.

### DETAILED DESCRIPTION

The present disclosure relates to methods and products for labelling, binding and detecting lipids.

Certain embodiments of the present disclosure are directed to methods and products that have one or more combinations of advantages. For example, some of the advantages of the embodiments disclosed herein include one or more of the following: a new and/or improved method of labelling or detecting lipids, including labelling or detecting specific types of lipids; lipid binding reagents that may be used for research purposes, and/or diagnostic or prognostic purposes; reagents that have the ability to label some intracellular structures, including endosomes, lysosomes autophagosomes and/or lipid droplets; reagents that may be used for labelling live cells; reagents that may be used for imaging live cells; reagents that may be used for diagnosis and/or prognosis; reagents that may detect cancerous cells; reagents that may be used to distinguish cancerous cells from non-cancerous cells; reagents that may be used to identify new biomarkers; improved methods of labelling or detecting lipids; to provide new methods for investigating cell biology; new methods and reagents for determining intracellular pH; to address one or more problems and/or to provide one or more advantages, or to provide a commercial alternative. Other advantages of certain embodiments of the present disclosure are also disclosed herein.

The present disclosure is based on the recognition that certain transitional metal based complexes have specificity for binding to lipids.

The complexes also have a variety of useful properties, including for example one or more of a small molecular size, they are adaptable to chemical modification, they exhibit emissive properties that are attributable to the core heavy metal, they unexpectedly have the ability to stain live cells and therefore may be used as live cell imaging agents, and they may be used to enable the visualization of pathogenic processes. They may also be used to determine intracellular pH, and in particular, the pH of intracellular structures and compartments where lipids are present.

One of the complexes identified interacts with lipid droplets in Drosophila fat body tissue, and relocates to acidic compartments during metamorphosis and starvation. The complex also interacts with autophagosomes following starvation. Another complex identified interacts with autophagosomes/acidic compartments, but not lipid droplets. The complexes interact with a variety of different lipids. The complexes also interact with cholesterol and/or cholesterol esters, providing the ability to explore different aspects of cholesterol biology. The complexes identified also interact with ecdysone, progesterone and steroid based detergents.

Certain embodiments of the present disclosure provide a method of labelling a lipid.

Certain embodiments of the present disclosure provide a method of labelling a lipid, the method comprising exposing the lipid to a complex comprising a transition metal carbonyl compound, a conjugated bidentate ligand and a tetrazolato compound and thereby labelling the lipid by binding the complex to the lipid.

In certain embodiments, the lipid comprises one or more of a polar lipid, a non-neutral lipid, a steroid, a sphingomyelin, a sphingosine, a neutral triglyceride, a monosialotetrahexosylganglioside, a phosphatidylethanolamine, a lysophosphatidic acid, a cholesterol, a cholesterol ester, a steroid compound, a steroid hormone, a progesterone and/or a derivative of any of the aforementioned.

In certain embodiments, the lipid comprises one or more of a polar lipid, a sphingomyelin, a sphingosine, a monosialotetrahexosylganglioside, a phosphatidylethanolamine, a lysophosphatidic acid, a cholesterol, a progesterone and/or a derivative of any of the aforementioned.

In certain embodiments, the lipid does not comprise one or more of a neutral lipid, a ceramide, a triglyceride, a fatty acid, a cholesterol ester and/or a derivative of any of the aforementioned.

In certain embodiments, the lipid comprises a steroid compound. Examples of steroid compounds include steroidal hormones (such as progesterone and oestrogen), a steroidal pro-hormone, such as ecydysone, or cholic acid derivatives such as CHAPS or deoxycholate. Other types of steroid compounds are contemplated.

In certain embodiments, the lipid comprises a cholesterol compound, being a group of compounds comprising four linked hydrocarbon rings forming a steroid structure.

In certain embodiments, the lipid comprises a non-esterified cholesterol. In certain embodiments, the lipid comprises an esterified cholesterol.

In certain embodiments, the lipid comprises a polar lipid. In certain embodiments, the lipid comprises a non-neutral lipid. In certain embodiments, the lipid comprises a non-long chain fatty acid.

In certain embodiments, the lipid is present in a non-biological sample. An example of a non-biological sample is a sample containing an artificially synthesized steroid, such as would be present in control and/or reference samples for steroid testing, such as in athletes. Other non-biological samples are contemplated.

In certain embodiments, the lipid is present in a biological sample. Examples of biological samples include a cell sample, a sample of live cells, a cell extract, a cell lysate, a cell-free sample, a sorted cell, a non-fixed cell, a fixed cell, a biopsy, a tissue sample, a bodily fluid sample, a blood sample, a urine sample, a saliva sample, a tissue section, mounted cells, a tissue sample, a drug doping sample and cells generally obtained or isolated from a subject, and/or an extract, component, derivative, processed form or purified form of any of the aforementioned. For example, the sample may be a blood or urine sample (or an extract, component, derivative or processed form thereof) from a human being tested for the presence of a steroid compound. It will be appreciated that in some embodiments, the term “cell” also refers to an extract, lysate, component, derivative, or a processed form of a cell.

In certain embodiments, the biological sample comprises a live cell.

In certain embodiments, the biological sample comprises a cell in vivo, an ex vivo cell and/or a cell in a biological fluid. In certain embodiments, the biological sample comprises a cell in vitro. It will be appreciated that the methods of the present disclosure may be performed in some embodiments wholly in vitro or ex vivo, or wholly in vivo.

In certain embodiments, the cell comprises one or more cells in a cell sample, one or more live cells, one or more fixed cells, one or more dead cells, one or more cells obtained from a subject, a sorted cell, a non-fixed cell, one or more cells in a biopsy, one or more cells in a tissue sample, one or more cells in a bodily fluid sample, one or more cells in a blood sample, one or more cells in a urine sample, one or more cells in a saliva sample, one or more cells in a tissue section, one or more cells mounted cells, one or more cells in a tissue sample, one or more cell in a drug doping sample and cells generally obtained or isolated from a subject. The cell may be in vitro, ex vivo or in vivo.

In certain embodiments, the cell may be a live cell, a fixed cell or a dead cell. Examples of cells are described herein. In certain embodiments, the cell is a cell associated with a disease, condition or state as described herein. In certain embodiments, the cell is a cell for which diagnostic and/or prognostic analysis is to be undertaken. In certain embodiments, the cell is obtained or isolated from a subject for which diagnostic or prognostic testing is to be undertaken. In certain embodiments, the cell is a cancerous cell or a non-cancerous cell.

In certain embodiments, the lipid is present in a biological fluid, such as blood, plasma, urine, milk, tears, saliva, and/or an extract, component, derivative, processed form or purified form thereof. Other fluids are contemplated.

In certain embodiments, the lipid comprises a natural lipid, a synthetic lipid, a non-naturally occurring lipid, a purified lipid, an isolated lipid, a non-cellular lipid, a cellular lipid, a mixture of lipids, or one or more lipids associated with a cellular structure. Other types of lipids are contemplated.

In certain embodiments, the lipid comprises a cellular lipid. In certain embodiments, the lipid comprises an intracellular lipid. In certain embodiments, the lipid comprises a lipid in a live cell, a fixed cell or a dead cell. In certain embodiments, the lipid comprises an intracellular lipid in a live cell.

In certain embodiments, the lipid comprises a free lipid, such as a steroid hormone in a urine, blood or plasma sample.

In certain embodiments, the lipid comprises a lipid in vitro, a lipid ex vivo and/or a lipid in vivo.

In certain embodiments, the lipid is associated with one or more intracellular structures.

In certain embodiments, the method comprises labelling a lipid in a non-biological setting, sample or environment.

In certain embodiments, the method comprises labelling a lipid in a biological setting, sample or environment. For example, the lipid may be present in a subject and labelling of the lipid occurs in vivo.

In certain embodiments, the method comprises labelling a cellular lipid. In certain embodiments, the method comprises labelling an intracellular lipid. In certain embodiments, the method comprises labelling a lipid in a live cell, a dead cell or a fixed cell. In certain embodiments, the method comprises labelling an intracellular lipid in a live cell.

The term “exposing”, and related terms such as “expose” and “exposure”, refers to contacting and/or treating a species (for example a lipid or a cell) with an effective amount of a complex as described herein. The term includes for example exposing a lipid in vitro to a complex as described herein, exposing a lipid in vivo to a complex as described herein, exposing a lipid ex vivo to a complex as described herein, and administering a complex as described herein to a subject so as to label lipids in vivo. Method for exposing species to a complex, including administration of agents to a subject, are known in the art. Methods for administering a complex to a subject to label a lipid or cell in vivo are known in the art.

Examples of subjects include humans, animals, such as livestock animals (eg a horse, a cow, a sheep, a goat, a pig), a domestic animal (eg a dog or a cat) and other types of animals such as monkeys, rabbits, mice and laboratory animals, and insects. Veterinary applications of the present disclosure are contemplated. Use of any of the aforementioned animal or insect models in the methods described herein is also contemplated, including methods of screening. In certain embodiments, the subject is human or animal subject.

Complexes (sometimes referred herein to as “probes”) comprising a transition metal carbonyl compound, a conjugated bidentate ligand and a tetrazolato compound may be synthesized by a method known in the art, for example as described in Wright P. J. et al (2013) “Ligand-Induced Structural, Photophysical, and Electrochemical Variations in Tricarbonyl Rhenium(I) Tetrazolato Complexes” Organometallics 32: 3728-3737 and Wright P. J. et al (2013) “Synthesis, Photophysical and Electrochemical Investigation of Dinuclear Tetrazolato-Bridged Rhenium Complexes” Organometallics 31: 7566-7578. It will be appreciated that the complexes as described herein include the complexes themselves, and/or a substituted form, an acceptable salt, a solvate, stereoisomer or a tautomer thereof.

In certain embodiments, the transition metal carbonyl compound comprises a transition metal ion in a complex comprising Re(I). Other transition metals are contemplated. Examples of other transition metal ions include Iridium and Ruthenium (Ir(III), and Ru(II)).

In certain embodiments, the transition metal carbonyl compound comprises a transition metal tricarbonyl compound. In certain embodiments, the transition metal carbonyl compound comprises a transition metal dicarbonyl compound. In certain embodiments, the transition metal carbonyl compound comprises a transition metal monocarbonyl compound. Methods of synthesis of transition metal carbonyl compounds are known in the art.

In certain embodiments, the conjugated bidentate ligand comprises a ligand that binds to the metal centre via at least one nitrogen atom. In certain embodiments, the conjugated bidentate ligand comprises a ligand that binds to the metal centre via two nitrogen atoms. In certain embodiments, the conjugated bidentate ligand comprises a ligand that binds to the metal centre via one nitrogen atom and a second atom, the second atom being oxygen, sulphur or phosphorous.

In certain embodiments, the conjugated bidentate ligand comprises an aromatic bidentate ligand. In certain embodiments, the conjugated bidentate ligand comprises a bidentate diimine ligand. In certain embodiments, the conjugated bidentate ligand comprises an aromatic diimine ligand.

In certain embodiments, the conjugated bidentate diimine ligand comprises a phenanthroline compound. In certain embodiments, the conjugated bidentate diimine ligand comprises a 1,10-phenanthroline and/or a substituted derivative thereof. In certain embodiments, the conjugated bidentate diimine ligand comprises a bipyridine compound, such as a 2,2′-bipyridine and/or a substituted derivative thereof.

In certain embodiments, the tetrazolato compound comprises an aryltetrazolate and/or a substituted derivative thereof. In certain embodiments, the tetrazolato compound comprises a heteroaryltetrazolate and/or a substituted derivative thereof. In certain embodiments, the tetrazolato compound comprises an alkyltetrazolate and/or a substituted derivative thereof. In certain embodiments, the tetrazolato compound comprises a cyanophenyltetrazolate and/or a substituted derivative thereof. In certain embodiments, the tetrazolato compound comprises a 4-cyanophenyltetrazolate and/or a substituted derivative thereof.

In certain embodiments, the tetrazolato compound comprises a pyridyltetrazolate and/or a substituted derivative thereof. In certain embodiments, the tetrazolato compound comprises a 3-pyridyltetrazolate and/or a substituted derivative thereof.

In certain embodiments, the tetrazolato compound comprises a methyl phenyl carbonate and/or a substituted derivative thereof. In certain embodiments, the tetrazolato compound comprises a 4-methyl phenyl carbonate and/or a substituted derivative thereof.

In certain embodiments, the complex comprises a metal ion complex according to the following formula:

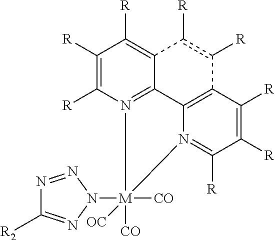

and/or an salt, solvate, tautomer or stereoisomer thereof,
wherein:
M is a transition metal ion;
R is H, hydroxyl, a halogen, optionally substituted alkyl, optionally substituted alkenyl, optionally substituted alkynyl, optionally substituted aryl, optionally substituted heteroalkyl, optionally substituted heterocycle, optionally substituted heteroaryl, optionally substituted alkoxy, optionally substituted aryloxy, optionally substituted heteroaryloxy, optionally substituted arylalkoxy, optionally substituted heteroarylalkoxy, an amine, an amide, a thiol, a phosphorus containing group, or a combination of any of the aforementioned; and
R₂ is an optionally substituted alkyl, optionally substituted alkenyl, optionally substituted alkynyl, optionally substituted aryl, optionally substituted heteroaryl, an optionally substituted aromatic group an optionally substituted aryl or heteroaryl ester, or a combination of any of the aforementioned.

Transition metal ions are as described herein. In certain embodiments, the transition metal ion comprises Re(I). Other transition metals are contemplated. Examples of other transition metal ions include Iridium and Ruthenium (Ir(III), and Ru(II)).

In certain embodiments, R₂ is an optionally substituted phenyl or an optionally substituted pyridyl group. In certain embodiments, R₂ is an optionally substituted cyanophenyl group. In certain embodiments, R₂ is an optionally substituted alkyl phenyl carbonate. In certain embodiments, R₂ is an optionally substituted methyl phenyl carbonate. In certain embodiments, R₂ is an optionally substituted 4-cyanophenyl group. In certain embodiments, R₂ is an optionally substituted 3-pyridyl group. In certain embodiments, R₂ is an optionally substituted 4-methyl phenyl carbonate group.

In certain embodiments, the complex comprises the following chemical structure:

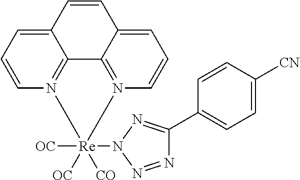

and/or a salt, solvate, tautomer or stereoisomer thereof.

In certain embodiments, the complex comprises the following chemical structure:

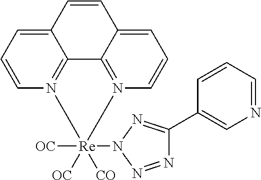

and/or a salt, solvate, tautomer or stereoisomer thereof.

In certain embodiments, the complex comprises a rhenium ion complex according to formula 1 and/or 2, and/or a salt, solvate, tautomer or stereoisomer thereof:

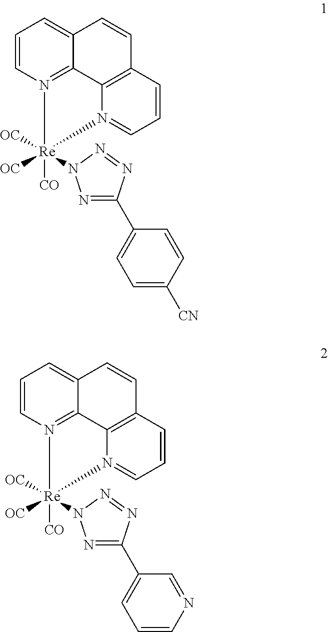

1: tricarbonyl-(4-cyanophenyltretrazolato)-phenanthroline-rhenium (I).
2: tricarbonyl-(3-pyridyltretrazolato) phenanthrolin rhenium (I).

In certain embodiments, the complex comprises the following chemical structure:

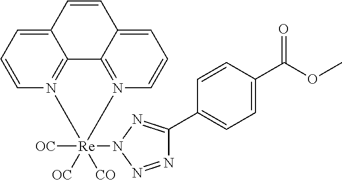

and/or a salt, solvate, tautomer or stereoisomer thereof.

In certain embodiments, the complex comprises the following chemical structure:

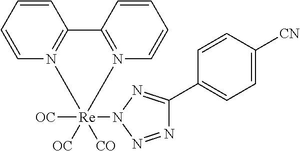

and/or a salt, solvate, tautomer or stereoisomer thereof.

In certain embodiments, the complex comprises the following chemical structure:

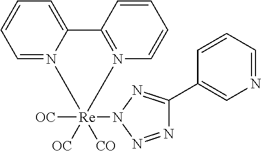

and/or a salt, solvate, tautomer or stereoisomer thereof.

In certain embodiments, the complex comprises the following chemical structure:

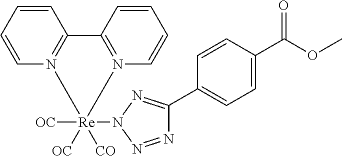

and/or a salt, solvate, tautomer or stereoisomer thereof.

In certain embodiments, the complex comprises tricarbonyl-(4-cyanophenyltretrazolato)-phenanthroline-rhenium (I) and the lipid comprises one or more of a polar lipid, a sphingomyelin, a sphingosine, a monosialotetrahexosylganglioside, a phosphatidylethanolamine, a lysophosphatidic acid, a cholesterol, a progesterone and/or a derivative of any of the aforementioned.

In certain embodiments, the complex comprises tricarbonyl-(4-cyanophenyltretrazolato)-phenanthroline-rhenium (I) and the lipid does not a neutral lipid, a ceramide, a triglycerides, a fatty acid, a cholesterol ester and/or a derivative of any of the aforementioned.

In certain embodiments, the complex comprises tricarbonyl-(3-pyridyltretrazolato) phenanthrolin rhenium (I) and the lipid comprises one or more of a polar lipid, a sphingomyelin, a sphingosine, a monosialotetrahexosylganglioside, a phosphatidylethanolamine, a lysophosphatidic acid, a cholesterol, a progesterone and/or a derivative of any of the aforementioned.

In certain embodiments, the complex comprises tricarbonyl-(3-pyridyltretrazolato) phenanthrolin rhenium (I) and the lipid does not comprise a neutral lipid, a ceramide, a triglycerides, a fatty acid, a cholesterol ester and/or a derivative of any of the aforementioned.

In certain embodiments, the complex comprises facial-Tricarbonyl (η¹(N2)-5-(methyl benzoate-4′-yl tetrazolato) η²-1,10-phenathroline Rhenium(I) and the lipid comprises one or more of a polar lipid, a cholesterol, a phosphatidylethanolamine, a sphingomyelin, a sphingosine, a monosialotetrahexosylganglioside, a neutral triglyceride and/or a derivative of any of the aforementioned.

In certain embodiments, the complex comprises facial-Tricarbonyl (η¹(N2)-5-(methyl benzoate-4′-yl tetrazolato) η²-2,2′-bipyridine Rhenium(I) and the lipid comprises one or more of a polar lipid, a cholesterol, a phosphatidylethanolamine, a sphingomyelin, a sphingosine, a monosialotetrahexosylganglioside, a neutral triglyceride and/or a derivative of any of the aforementioned.

In certain embodiments, the complex comprises facial-Tricarbonyl (η¹(N2)-5-cyanophen-4′-yl tetrazolato) η²-2,2′-bipyridine Rhenium(I) and the lipid comprises one or more of a polar lipid, a cholesterol, a phosphatidylethanolamine, a sphingomyelin, a sphingosine, a monosialotetrahexosylganglioside, a neutral triglyceride and/or a derivative of any of the aforementioned.

In certain embodiments, the method comprises labelling a free lipid. In certain embodiments, the method comprises labelling a lipid in a cell. In certain embodiments, the method comprises labelling a lipid in a cellular structure. In certain embodiments, the cellular structure comprises an intracellular structure. In certain embodiments, the cellular structure comprises a cellular structure containing lipid.

In certain embodiments, the cellular structure comprises one or more of an endosome, a lysosome, an autophagosome and a lipid droplet. In certain embodiments, the cellular structure comprises endoplasmic reticulum, Golgi, a plasma membrane. Other types of cellular structures are contemplated.

In certain embodiments, the complex comprises a 4-cyanophenyltetrazolato compound and the cellular structure comprises an autophagosome. In certain embodiments, the complex comprises tricarbonyl-(4-cyanophenyltretrazolato)-phenanthroline-rhenium (I) and the cellular structure comprises an autophagosome.

In certain embodiments, the complex comprises a 3-pyridyltetrazolato compound and the cellular structure comprises an endosome and/or a lysosome. In certain embodiments, the complex comprises tricarbonyl-(3-pyridyltretrazolato) phenanthroline rhenium (I) and the cellular structure comprises an endosome and/or a lysosome.

In certain embodiments, the method comprises labelling a lipid in a cellular structure. In certain embodiments, the cellular structure is a structure in a live cell, a dead, a non-fixed cell, a sorted cell, a fixed cell, a stained cell, an in vivo cell, an ex vivo cell or an in vitro cell. Other types of cells are contemplated, examples of which are described herein.

In certain embodiments, the cellular structure comprises one or more of an endosome, a lysosome, an autophagosome and a lipid droplet. In certain embodiments, the cellular structure comprises endoplasmic reticulum, Golgi, and a plasma membrane. Other types of cellular structures are contemplated.

In certain embodiments, the complex comprises a 4-cyanophenyltetrazolato compound and the cellular structure comprises an autophagosome. In certain embodiments, the complex comprises tricarbonyl-(4-cyanophenyltretrazolato)-phenanthroline-rhenium (I) and the cellular structure comprises an autophagosome

In certain embodiments, the complex comprises a 3-pyridyltetrazolato compound and the cellular structure comprises an endosome and/or a lysosome. In certain embodiments, the complex comprises tricarbonyl-(3-pyridyltretrazolato) phenanthroline rhenium (I) and the cellular structure comprises an endosome and/or a lysosome.

In certain embodiments, the method comprises exposing the lipid to two or more different complexes. For example, a cell may be exposed to two or more different complexes to label various types of selected lipids, such as in a cell.

In certain embodiments, the method is used to label or detect one or more of an endosome, lysosome, an autophagosome, a lipid droplet, to visualize an endosome, lysosome, an autophagosome and/or a lipid droplet, for intracellular imaging of a live cell, for detecting a disease, condition or state in a subject (such as a cancer), for identifying a subject suffering from, or susceptible to, a disease, condition or state (such as a cancer), for diagnosis and/or prognosis, to identify a subject suitable for treatment, to screen for the presence or absence of a cancer, for identifying a biomarker, to detect an anabolic steroid, to determine the level of an anabolic steroid and/or for testing for the presence or absence of an anabolic steroid. Other uses are contemplated.

Certain embodiments of the present disclosure provide a method of labelling or detecting a steroid compound.

Certain embodiments of the present disclosure provide a method of labelling a steroid compound by exposing the steroid compound to a complex as described herein.

Certain embodiments of the present disclosure provide a method of labelling a steroid compound, the method comprising exposing the steroid compound to a complex comprising a transition metal carbonyl compound, a conjugated bidentate ligand and a tetrazolato compound and thereby labelling the steroid compound by binding the complex to the steroid compound.

Certain embodiments of the present disclosure provide a method of labelling one or more of an endosome, a lysosome, an autophagosome and a lipid droplet.

Certain embodiments of the present disclosure provide a method of labelling one or more of an endosome, a lysosome, an autophagosome and a lipid droplet, the method comprising exposing one or more of an endosome, a lysosome, an autophagosome, and a lipid droplet to a complex comprising a transition metal carbonyl compound, a conjugated bidentate ligand and a tetrazolato compound and thereby labelling one or more of the endosome, the lysosome, the autophagosome and the lipid droplet by binding of the complex to one or more of the endosome, the lysosome, the autophagosome and the lipid droplet.

Certain embodiments of the present disclosure provide a method of labelling or detecting an endosome and/or a lysosome.

Certain embodiments of the present disclosure provide a method of labelling or detecting an endosome and/or a lysosome, the method comprising exposing the endosome and/or the lysosome to a complex as described herein.

Certain embodiments of the present disclosure provide a method of labelling an endosome and/or a lysosome, the method comprising exposing an endosome and/or a lysosome to a complex comprising a transition metal carbonyl compound, a conjugated bidentate ligand and a tetrazolato compound and thereby labelling the endosome and/or the lysosome by binding of the complex to the endosome and/or the lysosome.

Certain embodiments of the present disclosure provide a method of labelling or detecting an autophagosome.

Certain embodiments of the present disclosure provide a method of labelling or detecting an autophagosome, the method comprising exposing the autophagosome to a complex as described herein.

Certain embodiments of the present disclosure provide a method of labelling an autophagosome, the method comprising exposing an autophagosome to a complex comprising a transition metal carbonyl compound, a conjugated bidentate ligand and a tetrazolato compound and thereby labelling the autophagosome by binding of the complex to the autophagosome.

Certain embodiments of the present disclosure provide a method of labelling or detecting a lipid droplet.

Certain embodiments of the present disclosure provide a method of labelling or detecting a lipid droplet, the method comprising exposing the lipid droplet to a complex as described herein.

Certain embodiments of the present disclosure provide a method of labelling a lipid droplet, the method comprising exposing the lipid droplet to a complex comprising a transition metal carbonyl compound, a conjugated bidentate ligand and a tetrazolato compound and thereby labelling the lipid droplet by binding of the complex to the lipid droplet.

Certain embodiments of the present disclosure provide a method of labelling or detecting a cellular structure containing a lipid.

Certain embodiments of the present disclosure provide a method of labelling or detecting a cellular structure containing a lipid, the method comprising exposing the cellular structure to a complex as described herein.

Certain embodiments of the present disclosure provide a method of labelling a cellular structure containing a lipid, the method comprising exposing the cellular structure to a complex comprising a transition metal carbonyl compound, a conjugated bidentate ligand and a tetrazolato compound and thereby labelling the cellular structure by binding of the complex to the cellular structure.

Certain embodiments of the present disclosure provide a method of detecting a lipid.

Certain embodiments of the present disclosure provide a method of detecting a lipid, the method comprising binding to the lipid a complex as described herein.

Certain embodiments of the present disclosure provide a method of detecting a lipid, the method comprising exposing the lipid to a complex as described herein.

Certain embodiments of the present disclosure provide a method of detecting a lipid, the method comprising binding to the lipid a complex comprising a transition metal carbonyl compound, a conjugated bidentate ligand and a tetrazolato compound and detecting the complex bound to the lipid.

Transition metal ions that form part of a transitional metal carbonyl compound are as described herein. In certain embodiments, the transition metal ion comprises Re(I).

Transition metal carbonyl compounds are as described herein. In certain embodiments, the transition metal carbonyl compound comprises a transition metal tricarbonyl compound.

Conjugated bidentate ligands are as described herein. In certain embodiments, the conjugated bidentate ligand comprises a bidentate diimine ligand.

In certain embodiments, the conjugated bidentate ligand comprises a phenanthroline compound. In certain embodiments, the phenanthroline compound comprises a 1,10-phenanthroline and/or a substituted derivative thereof.

In certain embodiments, the conjugated bidentate ligand comprises a bipyridine ligand. In certain embodiments, the conjugated bidentate diimine ligand comprises a 2,2′-bipyridine and/or a substituted derivative thereof.

Tetrazolato compounds are as described herein. In certain embodiments, the tetrazolato compound comprises a cyanophenyltetrazolate and/or a substituted derivative thereof. In certain embodiments, the tetrazolato compound comprises a 4-cyanophenyltetrazolate and/or a substituted derivative thereof.

In certain embodiments, the tetrazolato compound comprises a pyridyltetrazolate and/or substituted derivative thereof. In certain embodiments, the tetrazolato compound comprises a 3-pyridyltetrazolate and/or substituted derivative thereof.

In certain embodiments, the tetrazolato compound comprises a methyl phenyl carbonate and/or substituted derivative thereof. In certain embodiments, the tetrazolato compound comprises a 4-methyl phenyl carbonate and/or substituted derivative thereof.

Lipids are as described herein.

In certain embodiments, the lipid comprises one or more of a polar lipid, a non-neutral lipid, a steroid, a sphingomyelin, a sphingosine, a neutral triglyceride, a monosialotetrahexosylganglioside, a phosphatidylethanolamine, a lysophosphatidic acid, a cholesterol, a cholesterol ester, a steroid compound, a steroid hormone, a progesterone and/or a derivative of any of the aforementioned.

In certain embodiments, the lipid comprises one or more of a polar lipid, a non-neutral lipid, a steroid, a sphingomyelin, a sphingosine, a neutral triglyceride, a monosialotetrahexosylganglioside, a phosphatidylethanolamine, a lysophosphatidic acid, a cholesterol, a cholesterol ester, a steroid compound, a steroid hormone, a progesterone and/or a derivative of any of the aforementioned.

In certain embodiments, the lipid comprises one or more of a polar lipid, a sphingomyelin, a sphingosine, a monosialotetrahexosylganglioside, a phosphatidylethanolamine, a lysophosphatidic acid, a cholesterol, a progesterone and/or a derivative of any of the aforementioned.

In certain embodiments, the lipid does not comprise a neutral lipid, a ceramide, a triglycerides, a fatty acid, a cholesterol ester and/or a derivative of any of the aforementioned.

In certain embodiments, the lipid comprises a steroid compound.

In certain embodiments, the lipid comprises a cholesterol. In certain embodiments, the cholesterol comprises an esterified cholesterol. In certain embodiments, the cholesterol comprises a non-esterified cholesterol.

In certain embodiments, the complex comprises tricarbonyl-(4-cyanophenyltretrazolato)-phenanthroline-rhenium (I) and the lipid comprises one or more of a polar lipid, a sphingomyelin, a sphingosine, a monosialotetrahexosylganglioside, a phosphatidylethanolamine, a lysophosphatidic acid, a cholesterol, a progesterone and/or a derivative of any of the aforementioned.

In certain embodiments, the complex comprises tricarbonyl-(4-cyanophenyltretrazolato)-phenanthroline-rhenium (I) and the lipid does not comprise a neutral lipid, a ceramide, a triglycerides, a fatty acid, a cholesterol ester and/or a derivative of any of the aforementioned.

In certain embodiments, the complex comprises tricarbonyl-(3-pyridyltretrazolato) phenanthrolin rhenium (I) and the lipid comprises one or more of a polar lipid, a sphingomyelin, a sphingosine, a monosialotetrahexosylganglioside, a phosphatidylethanolamine, a lysophosphatidic acid, a cholesterol, a progesterone and/or a derivative of any of the aforementioned.

In certain embodiments, the complex comprises tricarbonyl-(3-pyridyltretrazolato) phenanthrolin rhenium (I) and the lipid does not comprise a neutral lipid, a ceramide, a triglycerides, a fatty acid, a cholesterol ester and/or a derivative of any of the aforementioned.

In certain embodiments, the complex comprises facial-Tricarbonyl (η¹(N2)-5-(methyl benzoate-4′-yl tetrazolato) η²-1,10-phenathroline Rhenium(I) and the lipid comprises one or more of a polar lipid, a cholesterol, a phosphatidylethanolamine, a sphingomyelin, a sphingosine, a monosialotetrahexosylganglioside, a neutral triglyceride and/or a derivative of any of the aforementioned.

In certain embodiments, the complex comprises facial-Tricarbonyl (η1 (N2)-5-(methyl benzoate-4′-yl tetrazolato) η2-2,2′-bipyridine Rhenium(I) and the lipid comprises one or more of a polar lipid, a cholesterol, a phosphatidylethanolamine, a sphingomyelin, a sphingosine, a monosialotetrahexosylganglioside, a neutral triglyceride and/or a derivative of any of the aforementioned.

In certain embodiments, the complex comprises facial-Tricarbonyl (η¹(N2)-5-cyanophen-4′-yl tetrazolato) η²-2,2′-bipyridine Rhenium(I) and the lipid comprises one or more of a polar lipid, a cholesterol, a phosphatidylethanolamine, a sphingomyelin, a sphingosine, a monosialotetrahexosylganglioside, a neutral triglyceride and/or a derivative of any of the aforementioned.

In certain embodiments, the method comprises binding the complex to a lipid in a cellular structure. In certain embodiments, the cellular structure comprises one or more of an endosome, a lysosome and an autophagosome. In certain embodiments, the cellular structure comprises endoplasmic reticulum, Golgi, a plasma membrane, and lipid droplets.

In certain embodiments, the method comprises binding the complex to a cellular structure containing a lipid.

Examples of cellular structures, intracellular structures and cell compartments are as described herein.

In certain embodiments, the complex comprises a 4-cyanophenyltetrazolato compound and the cellular structure comprises an autophagosome.

In certain embodiments, the complex comprises a 3-pyridyltetrazolato compound and the cellular structure comprises an endosome and/or a lysosome.

In certain embodiments, the lipid is present in a non-biological sample.

In certain embodiments, the lipid comprises a free lipid.

In certain embodiments, the lipid is present in a biological sample. In certain embodiments, the biological sample comprises a cell sample, a sample of live cells, a cell extract, a non-fixed cell, a sorted cell, a fixed cell, a biopsy, a bodily fluid sample, a blood sample, a urine sample, a saliva sample and/or an extract, component, derivative, processed form or purified form of any of the aforementioned.

In certain embodiments, the lipid is present in a cell. Examples of cells are as described herein. In certain embodiments, the lipid is present in a cancerous cell or a non-cancerous cell.

In certain embodiments, the method comprises detecting an intracellular lipid. In certain embodiments, the method comprises detecting an intracellular lipid in a live cell.

In certain embodiments, the method comprises binding two or more different complexes to the lipid.

Certain embodiments of the present disclosure provide use of a complex as described herein to label or detect a lipid, to label or detect a cellular structure as described herein, or detect or label cells as described herein.

Certain embodiments of the present disclosure provide a method of detecting a steroid compound.

Certain embodiments of the present disclosure provide a method of detecting a steroid compound by binding a complex as described herein to the steroid compound.

Certain embodiments of the present disclosure provide a method of detecting a steroid compound, the method comprising binding to the steroid compound a complex comprising a transition metal carbonyl compound, a conjugated bidentate ligand and a tetrazolato compound and detecting the complex bound to the steroid compound.

Certain embodiments of the present disclosure provide a method of detecting an endosome and/or a lysosome. Certain embodiments of the present disclosure provide a method of detecting an endosome and/or a lysosome by binding a complex to the endosome and/or the lysosome a complex as described herein.

Certain embodiments of the present disclosure provide a method of detecting an endosome and/or a lysosome, the method comprising binding to an endosome and/or a lysosome a complex comprising a transition metal carbonyl compound, a conjugated bidentate ligand and a 3-pyridyltetrazolato compound and thereby detecting the endosome and/or the lysosome by detecting the complex bound to the endosome and/or the lysosome.

Certain embodiments of the present disclosure provide a method of detecting an autophagosome.

Certain embodiments of the present disclosure provide a method of detecting an autophagosome by binding to the autophagosome a complex as described herein.

Certain embodiments of the present disclosure provide a method of detecting an autophagosome, the method comprising binding to an autophagosome a complex comprising a transition metal carbonyl compound, a conjugated bidentate ligand and a 4-cyanophenyltetrazolato compound and detecting the autophagosome by detecting the complex bound to the autophagosome.

Certain embodiments of the present disclosure provide a method of detecting a lipid droplet.

Certain embodiments of the present disclosure provide a method of detecting a lipid droplet by binding to the cellular structure a complex as described herein.

Certain embodiments of the present disclosure provide a method of detecting a lipid droplet, the method comprising binding to a lipid droplet a complex comprising a transition metal carbonyl compound, a conjugated bidentate ligand and a tetrazolato compound and detecting the lipid droplet by detecting the complex bound to the lipid droplet.

Certain embodiments of the present disclosure provide a method of detecting a cellular structure containing lipid.

Certain embodiments of the present disclosure provide a method of detecting a cellular structure containing lipid by binding to the cellular structure a complex as described herein.

Certain embodiments of the present disclosure provide a method of detecting a cellular structure containing lipid, the method comprising binding to a cellular structure containing lipid a complex comprising a transition metal carbonyl compound, a conjugated bidentate ligand and a tetrazolato compound and detecting the cellular structure by detecting the complex bound to the cellular structure containing lipid.

Certain embodiments of the present disclosure provide a method of intracellular imaging of a cell. Certain embodiments of the present disclosure provide a method of intracellular imaging of a cell by exposing the cell to a complex as described herein.

Certain embodiments of the present disclosure provide a method of intracellular imaging of a cell, the method comprising exposing a cell to a complex comprising a transition metal carbonyl compound, a conjugated bidentate ligand and a tetrazolato compound and intracellularly imaging lipids in the cell bound to the complex.

Examples of cells are as described herein. In certain embodiments, the cell is a cell associated with a disease, condition or state as described herein. In certain embodiments, the cell is a cancerous cell or a non-cancerous cell.

In certain embodiments, the cell comprises a live cell.

Certain embodiments of the present disclosure provide a method of intracellular imaging of a live cell. Certain embodiments of the present disclosure provide a method of intracellular imaging of a live cell by exposing the cell to a complex as described herein.

Certain embodiments of the present disclosure provide a method of intracellular imaging of a live cell, the method comprising exposing a live cell to a complex comprising a transition metal carbonyl compound, a conjugated bidentate ligand and a tetrazolato compound and intracellularly imaging lipids in the live cell bound to the complex.

In certain embodiments, the cell comprises a cell in vivo. In certain embodiments, the cell comprises a cell ex vivo. In certain embodiments, the cell comprises a cell in a biological sample. In certain embodiments, the cell comprises a cell in vitro.

In certain embodiments, the cell is a cell associated with a disease, condition or state as described herein.

In certain embodiments, the cell is a cell from a human, an animal, an insect, a livestock animal (such as a horse, a cow, a sheep, a goat, a pig), a domestic animal (such as a dog or a cat) and other types of animals such as monkeys, rabbits, mice and laboratory animals. Veterinary applications of the present disclosure are contemplated. Use of any of the aforementioned animals as animal models in the methods is also contemplated.

Methods for administering complexes to an organism are known in the art.

Certain embodiments of the present disclosure provide use of a complex as described herein as an intracellular imaging agent.

Certain embodiments of the present disclosure provide an intracellular imaging agent.

Certain embodiments of the present disclosure provide an intracellular imaging agent, the agent comprising a complex comprising a transition metal carbonyl compound, a conjugated bidentate ligand and a tetrazolato compound.

Complexes comprising a transition metal carbonyl compound, a conjugated bidentate ligand and a tetrazolato compound are as described herein.

Certain embodiments of the present disclosure provide a composition for intracellular imaging, the composition comprising a transition metal carbonyl compound, a conjugated bidentate ligand and a tetrazolato compound are as described herein.

In certain embodiments, the composition comprises DMSO.

For example, a stock solution of the complex in DMSO may be prepared and the stock solution subsequently diluted in PBS for exposing to lipids, cells and cell structures.

Certain embodiments of the present disclosure provide a method of intracellular imaging, the method comprising use of an intracellular imaging agent, and/or intracellular imaging composition as described herein.

Certain embodiments of the present disclosure provide a method of intracellularly imaging a cell.

Certain embodiments of the present disclosure provide a method of intracellularly imaging a cell, the method comprising exposing a cell to an intracellular imaging agent as described herein and intracellularly imaging lipids in the cell bound to the agent.

Certain embodiments of the present disclosure provide a kit for performing a method as described herein.

Certain embodiments of the present disclosure provide a kit for intracellular imaging of cells, the kit comprising a complex comprising a transition metal carbonyl compound, a conjugated bidentate ligand and a tetrazolato compound.

In certain embodiments, the kit further comprises instructions.

For example, the kit may also include instructions for using the complex, instructions for exposing the cells to the complex and/or instructions for imaging the cells.

In certain embodiments, the kit further comprises one or more other reagents for imaging of cells, including enhancers, stabilisers and controls.

In certain embodiments, the kit comprises DMSO and/or the complex in DMSO.

Certain embodiments of the present disclosure provide a kit for intracellular imaging of live cells, the kit comprising a complex comprising a transition metal carbonyl compound, a conjugated bidentate ligand and a tetrazolato compound.

In certain embodiments, the kit further comprises instructions.

For example, the kit may also include instructions for using the complex, instructions for exposing the live cells to the complex and/or instructions for imaging the cells.

In certain embodiments, the kit further comprises one or more other reagents for imaging of live cells, including enhancers, stabilisers and controls.

In certain embodiments, the kit comprises DMSO, the complex in DMSO and/or the complex in DMSO being further diluted into another medium, such as PBS.

Certain embodiments of the present disclosure provide a kit for labelling lipids, the kit comprising a complex comprising a transition metal carbonyl compound, a conjugated bidentate ligand and a tetrazolato compound.

In certain embodiments, the kit further comprises instructions. For example, the kit may also include instructions for labelling lipids, instructions for exposing the cells to the complex and/or instructions for detecting and/or visualising the label.

In certain embodiments, the kit further comprises one or more other reagents for labelling, including enhancers, stabilisers and controls.

In certain embodiments, the kit comprises DMSO and/or the complex in DMSO.

Certain embodiments of the present disclosure provide a kit for labelling intracellular structures containing lipid, the kit comprising a complex comprising a transition metal carbonyl compound, a conjugated bidentate ligand and a tetrazolato compound.

In certain embodiments, the kit further comprises instructions. For example, the kit may also include instructions for labelling intracellular structures, instructions for exposing intracellular structures and/or cells to the complex, and/or instructions for detecting and/or visualising the label.

In certain embodiments, the kit further comprises one or more other reagents for labelling, including enhancers, stabilisers and controls.

In certain embodiments, the kit comprises DMSO and/or complex in DMSO.

Certain embodiments of then present disclosure provide an isolated lipid bound to a complex as described herein, which may for example, be useful as a reagent in a kit, or as a positive control or reference sample.

The term “isolated” refers to a species, such as a lipid, that has been separated from its natural environment. Lipids and complexes are as described herein.

Certain embodiments of the present disclosure provide an isolated lipid bound to a complex comprising a transition metal carbonyl compound, a conjugated bidentate ligand and a tetrazolato compound transition metal carbonyl compound.

Certain embodiments of the present disclosure provide an isolated steroid compound bound to a complex comprising a transition metal carbonyl compound, a conjugated bidentate ligand and a tetrazolato compound.

Steroids are as described herein. In certain embodiments, the steroid comprises a cholesterol.

Certain embodiments of the present disclosure provide a method of identifying a cellular structure using a complex as described herein.

In certain embodiments, the cellular structure comprises an intracellular structure. In certain embodiments, the cellular structure comprises a subcellular structure and/or a cellular compartment.

Examples of cellular structures include a subcellular structure, a cellular compartment, endosomes, lysosomes and/or autophagosomes, endoplasmic reticulum, Golgi, a plasma membrane, and lipid droplets.

Certain embodiments of the present disclosure provide a method of identifying a cellular structure as an autophagosome, the method comprising exposing the cellular structure to a complex comprising a transition metal carbonyl compound, a conjugated bidentate ligand and a 4-cyanophenyltetrazolato compound and identifying the cellular structure as an autophagosome on the basis of the labelling of the structure with the complex.

Certain embodiments of the present disclosure provide a method of identifying a cellular structure as an endosome and/or a lysosome, the method comprising binding to an endosome and/or a lysosome a complex comprising a transition metal carbonyl compound, a conjugated bidentate ligand and a 3-pyridyltetrazolato compound and identifying the endosome and/or the lysosome on the basis of the labelling of the structure with the complex.

Certain embodiments of the present disclosure provide use of a complex as described herein to detect a disease, condition or state.

Certain embodiments of the present disclosure provide a method of detecting a disease, condition or state using a complex as described herein. For example, changes in lipid biology may be indicative of certain pathological processes, such a cancer

Certain embodiments of the present disclosure provide a method of detecting a disease, condition or state in a subject associated with altered or dysfunctional endosomal functionality, lysosomal functionality and/or autophagy, the method comprising labelling one or more of endosomes, lysosome, autophagosomes and lipid droplets from the subject with a complex comprising a transition metal carbonyl compound, a conjugated bidentate ligand and a tetrazolato compound and detecting the disease, condition or state on the basis of the one or more endosomes, lysosomes, autophagosomes and lipid droplets so labelled.

In certain embodiments, the disease, condition or state comprises a disease, condition or state associated with altered or dysfunctional autophagy.

In certain embodiments, the disease, condition or state comprises a disease, condition or state associated with altered or dysfunctional lipid intake, metabolism, processing, biogenesis or accumulation.

Examples of diseases, conditions or states include heart disease, obesity, cancer, neurodegenerative diseases, diabetes, muscular dystrophies, lysosomal storage disorders, metabolic disorders, pulmonary diseases, diseases, conditions or states associated with altered or dysfunctional development and/or remodelling, a steroid lipid linked pathology, and oogenesis.

Examples of subjects include humans, animals, such as livestock animals (eg a horse, a cow, a sheep, a goat, a pig), a domestic animal (eg a dog or a cat) and other types of animals such as monkeys, rabbits, mice and laboratory animals, and insects. Other types of subjects are contemplated.

In certain embodiments, the method comprises intracellular imaging. In certain embodiments, the method comprises intracellular imaging of cells. In certain embodiments, the intracellular imaging comprises intracellular imaging of live cells.

In certain embodiments, the method comprises intracellular imaging in vivo. In certain embodiments. In certain embodiments, the method comprises intracellular imaging ex vivo. In certain embodiments, the method comprises intracellular imaging in vitro.

In certain embodiments, the method comprises obtaining one or more cells from the subject and exposing the cells so obtained to the complex. In certain embodiments, the method comprises exposing cells isolated from a subject to the complex. Methods for obtaining cells are known in the art. For example, one or more cells may be obtained by taking a biopsy from the subject and the cells or lipids in the biopsy (or a processed form of the biopsy) labelled with a complex as described herein.

Certain embodiments of the present disclosure provide a method of detecting a disease, condition or state in a subject associated with altered or dysfunctional endosomal functionality, lysosomal functionality and/or autophagy, the method comprising exposing one or more cells (and/or components of cells) from the subject to a complex comprising a transition metal carbonyl compound, a conjugated bidentate ligand and a tetrazolato compound to intracellularly label the one or more cells and detecting the disease, condition or state on the basis of the cells so labelled.

In certain embodiments, the method comprises intracellular imaging. In certain embodiments, the intracellular imaging comprises intracellular imaging of live cells.

In certain embodiments, the method comprises obtaining one or more cells from the subject and exposing the cells so obtained to the complex. In certain embodiments, the method comprises exposing one or more cells isolated from a subject to the complex.

Certain embodiments of the present disclosure provide a method of detecting a disease, condition or state in a subject associated with altered or dysfunctional lipid intake, metabolism, processing, biogenesis and/or accumulation, the method comprising exposing one or more cells from the subject to a complex comprising a transition metal carbonyl compound, a conjugated bidentate ligand and a tetrazolato compound to intracellularly label the one or more cells and detecting the disease, condition or state on the basis of the cells so labelled.

In certain embodiments, the method comprises intracellular imaging. In certain embodiments, the method comprises intracellular imaging in vivo, ex vivo or in vitro. In certain embodiments, the intracellular imaging comprises intracellular imaging of live cells.

In certain embodiments, the method comprises obtaining one or more cells from the subject and exposing the cells so obtained to the complex. In certain embodiments, the method comprises exposing one or more cells isolated from a subject to the complex.

Certain embodiments of the present disclosure provide a method of detecting a disease, condition or state in a subject associated with altered or dysfunctional lipid functionality, the method comprising exposing one or more cells from the subject to a complex comprising a transition metal carbonyl compound, a conjugated bidentate ligand and a tetrazolato compound to label the one or more cells and detecting the disease, condition or state on the basis of the cells so labelled.

In certain embodiments, the method comprises intracellular imaging. In certain embodiments, the method comprises intracellular imaging in vivo, ex vivo or in vitro. In certain embodiments, the intracellular imaging comprises intracellular imaging of live cells.

In certain embodiments, the method comprises obtaining one or more cells from the subject and exposing the cells so obtained to the complex. In certain embodiments, the method comprises exposing one or more cells isolated from a subject to the complex.

Certain embodiments of the present disclosure provide use of a complex as described herein to identify a subject suffering from, or susceptible to, a disease, condition or state, such a cancer

Certain embodiments of the present disclosure provide a method of identifying a subject suffering from, or susceptible to, a disease, condition or state, such as a cancer

In certain embodiments, the subject is suffering from, or susceptible to a disease, condition or state associated with altered or dysfunctional endosomal functionality, lysosomal functionality and/or autophagy. In certain embodiments, the subject is suffering from, or susceptible to a disease, condition or state in a subject associated with altered or dysfunctional lipid intake, lipid metabolism, lipid processing, lipid biogenesis and/or lipid accumulation.

In certain embodiments, the subject is suffering from, or susceptible to a cancer.

In certain embodiments, the method comprises intracellular imaging. In certain embodiments, the method comprises intracellular imaging in vivo, ex vivo or in vitro. In certain embodiments, the intracellular imaging comprises intracellular imaging of live cells.

In certain embodiments, the method comprises obtaining one or more cells from the subject and exposing the cells so obtained to the complex. In certain embodiments, the method comprises exposing one or more cells isolated from a subject to the complex.

Certain embodiments of the present disclosure provide a method of identifying a subject suffering from, or susceptible to, a disease, condition or state in a subject associated with altered or dysfunctional endosomal functionality, lysosomal functionality and/or autophagy, the method comprising labelling one or more of endosomes, lysosome, autophagosomes and lipid droplets from the subject with a complex comprising a transition metal carbonyl compound, a conjugated bidentate ligand and a tetrazolato compound and identifying the subject as suffering from, or being susceptible to, the disease, condition or state on the basis of the one or more endosomes, lysosome, autophagosomes and lipid droplets so labelled.

Certain embodiments of the present disclosure provide a method of identifying a subject suffering from, or susceptible to, a disease, condition or state in a subject associated with altered or dysfunctional lipid intake, metabolism, processing, biogenesis and/or accumulation, the method comprising exposing one or more cells from the subject to a complex comprising a transition metal carbonyl compound, a conjugated bidentate ligand and a tetrazolato compound to intracellularly label the one or more cells and identifying the subject as suffering from, or being susceptible to, the disease, condition or state on the basis of the cells (and/or components) so labelled.

Certain embodiments of the present disclosure provide a method of identifying a subject suffering from, or susceptible to, a disease, condition or state in a subject associated with altered or dysfunctional endosomal functionality, lysosomal functionality and/or autophagy, the method comprising exposing one or more cells from the subject to a complex comprising a transition metal carbonyl compound, a conjugated bidentate ligand and a tetrazolato compound to intracellularly label the one or more cells and identifying the subject suffering from, or susceptible to, the disease, condition or state on the basis of the cells so labelled.

Certain embodiments of the present disclosure provide a method of identifying a subject suffering from, or susceptible to, a disease, condition or state in a subject associated with altered or dysfunctional lipid intake, metabolism, processing, biogenesis and/or accumulation, the method comprising exposing one or more cells from the subject to a complex comprising a transition metal carbonyl compound, a conjugated bidentate ligand and a tetrazolato compound to intracellularly label the one or more cells and identifying the subject suffering from, or susceptible to, the disease, condition or state on the basis of the cells so labelled.

Certain embodiments of the present disclosure provide screening methods for identifying a complex for labelling, detecting or binding to a lipid.

Such methods may be used to screen new reagents for research and diagnostic/prognostic purposes. For example, complexes so identified may be used for diagnostic imaging for lipid linked pathologies, such as heart disease, obesity and cancer.

Certain embodiments of the present disclosure provide a method of identifying a complex for labelling a lipid, the method comprising:
providing a candidate complex comprising a transition metal carbonyl compound, a conjugated bidentate ligand and a tetrazolato compound;
determining the ability of the candidate complex to bind to a lipid; and
identifying the candidate complex as a complex for labelling a lipid.

Certain embodiments of the present disclosure provide a method of identifying a complex for labelling a lipid, the method comprising:
providing a candidate complex comprising a transition metal carbonyl compound, a conjugated bidentate ligand and a tetrazolato compound;
determining the ability of the candidate complex to bind to a lipid; and
identifying the candidate complex as a complex for labelling a lipid on the basis of binding of the complex to the lipid.

Method for assessing the ability of a complex to bind to, or label, a lipid are known in the art. In

Candidate complexes comprising a transition metal carbonyl compound, a conjugated bidentate ligand and a tetrazolato compound are as described herein. Methods for determining the ability of a candidate complex to bind to or label a lipid are known in the art, such as in vitro methods such as 2D NMR and BioCore analysis or methods as described herein for example involving binding intracellular lipids. A variety of lipid types may be screened, for example steroid lipids. Lipids may tested in vitro, in vivo, or in an animal or insect model for example.

Certain embodiments of the present disclosure provide a method of identifying a complex for binding a lipid, the method comprising:
providing a candidate complex comprising a transition metal carbonyl compound, a conjugated bidentate ligand and a tetrazolato compound;
determining the ability of the candidate complex to bind to a lipid; and
identifying the candidate complex as a complex for binding a lipid.

Certain embodiments of the present disclosure provide a method of identifying a complex for binding a lipid, the method comprising:
providing a candidate complex comprising a transition metal carbonyl compound, a conjugated bidentate ligand and a tetrazolato compound;
determining the ability of the candidate complex to bind to a lipid; and
identifying the candidate complex as a complex for binding a lipid on the basis of the complex binding to the lipid.

Certain embodiments of the present disclosure provide a method of identifying a complex for detecting a lipid, the method comprising:
providing a candidate complex comprising a transition metal carbonyl compound, a conjugated bidentate ligand and a tetrazolato compound;
determining the ability of the candidate complex to bind to a lipid; and
identifying the candidate complex as a complex for detecting a lipid.

Certain embodiments of the present disclosure provide a method of identifying a complex for detecting a lipid, the method comprising:
providing a candidate complex comprising a transition metal carbonyl compound, a conjugated bidentate ligand and a tetrazolato compound;
determining the ability of the candidate complex to bind to a lipid; and
identifying the candidate complex as a complex for detecting a lipid on the basis of the complex binding to the lipid.

Certain embodiments of the present disclosure provide screening methods for identifying compounds for intracellular imaging of a cell.

Certain embodiments of the present disclosure provide a method of identifying a compound for intracellular imaging of a cell, the method comprising:
providing a candidate complex comprising a transition metal carbonyl compound, a conjugated bidentate ligand and a tetrazolato compound;
determining the ability of the candidate complex to bind to a lipid in the cell; and
identifying the candidate complex as a compound for labelling a lipid.

Certain embodiments of the present disclosure provide a method of identifying a compound for intracellular imaging of a cell, the method comprising:
providing a candidate complex comprising a transition metal carbonyl compound, a conjugated bidentate ligand and a tetrazolato compound;
determining the ability of the candidate complex to bind to a lipid in the cell; and
identifying the candidate complex as a compound for labelling a lipid on the basis of the complex binding to a lipid in a cell.

In certain embodiments, the cell is a live cell.

Certain embodiments of the present disclosure provide a method of identifying diagnostic or prognostic markers using a complex as described herein.

Certain embodiments of the present disclosure provide a method of identifying a diagnostic or prognostic marker, the method comprising using a complex comprising a transition metal carbonyl compound, a conjugated bidentate ligand and a tetrazolato compound to identify a molecule associated with altered or dysfunctional endosomal function, lysosomal function, autophagy and altered or dysfunctional lipid intake, metabolism, processing, biogenesis and/or accumulation.

In certain embodiments, the molecule comprises a lipid molecule.

Certain embodiments of the present disclosure provide a method of identifying a diagnostic or prognostic marker, the method comprising using a complex comprising a transition metal carbonyl compound, a conjugated bidentate ligand and a tetrazolato compound to identify a lipid molecule associated with altered or dysfunctional endosomal function, lysosomal function, autophagy, and altered or dysfunctional lipid intake, metabolism, processing, biogenesis and/or accumulation.

For example, lipids may be screened using a complex as described herein to determine whether specific lipids are indicative of a specific pathology, and thereby identify new diagnostic and/or prognostic markers.

Certain embodiments of the present disclosure provide a method of identifying a cancerous cell.

Certain embodiments of the present disclosure provide a method of identifying a cancerous cell on the basis of increased labelling of the cell by a complex as described herein.

Certain embodiments of the present disclosure provide a method of identifying a cancerous cell, the method comprising exposing a cell to a complex comprising a transition metal carbonyl compound, a conjugated bidentate ligand and a tetrazolato compound and identifying the cell as a cancerous cell by increased labelling of the cell by the complex.

In certain embodiments, the increased labelling of the cell is as compared to a non-cancerous cell.

Examples of cells are as described herein.

In certain embodiments, the cell is present in a biological sample. Examples of biological samples include a cell sample, a sample of live cells, a cell extract, a cell lysate, a cell-free sample, a sorted cell, a non-fixed cell, a fixed cell, a biopsy, a tissue sample, a bodily fluid sample, a blood sample, a urine sample, a saliva sample, a tissue section, mounted cells, a tissue sample, a drug doping sample and cells generally obtained or isolated from a subject, and/or an extract, component, derivative, processed form or purified form of any of the aforementioned.

In certain embodiments, the cell comprises a live cell.

In certain embodiments, the cell comprises a cell in vivo, an ex vivo cell, a cell in vitro, a cell in a biopsy and/or a cell in a biological fluid. The cell may for example be a live cell, a fixed cell or a dead cell.

In certain embodiments, the method comprises intracellular imaging of the cell. In certain embodiments, the intracellular imaging comprises intracellular imaging of a live cell.

In certain embodiments, the method comprises obtaining one or more cells from a subject and exposing the cells so obtained to the complex. In certain embodiments, the method comprises exposing one or more cells obtained from a subject to the complex.

In certain embodiments, the method is used to detect the presence of cancerous cells, or a cancer, in a subject. In certain embodiments, the method is used to detect the presence or absence of a cancer in a subject. In certain embodiments, the method is used to screen for the presence or absence of a cancer in a subject.

Certain embodiments of the present disclosure provide a method of screening for a cancerous cell.

Certain embodiments of the present disclosure provide a method of screening for a cancerous cell, the method comprising exposing a cell to a complex comprising a transition metal carbonyl compound, a conjugated bidentate ligand and a tetrazolato compound and identifying the cell as a cancerous cell or a non-cancerous cell on the basis of the labelling of the cell by the complex.

In certain embodiments, increased labelling of the cell is indicative that the cell is a cancerous cell. In certain embodiments, the absence of increased labelling is indicative that the cell is a non-cancerous cell.

Certain embodiments of the present disclosure provide a method of screening for a cancerous cell, the method comprising exposing a cell to a complex comprising a transition metal carbonyl compound, a conjugated bidentate ligand and a tetrazolato compound and identifying the cell as a cancerous cell or a non-cancerous cell on the basis of the labelling of the cell by the complex, wherein an increased labelling of the cell is indicative that the cell is a cancerous cell.

In certain embodiments, the increased labelling of the cell is as compared to a non-cancerous cell.

Examples of cells are as described herein.

In certain embodiments, the cell is present in a biological sample. Examples of biological samples include a cell sample, a sample of live cells, a cell extract, a cell lysate, a cell-free sample, a sorted cell, a non-fixed cell, a fixed cell, a biopsy, a tissue sample, a bodily fluid sample, a blood sample, a urine sample, a saliva sample, a tissue section, mounted cells, a tissue sample, a drug doping sample and cells generally obtained or isolated from a subject, and/or an extract, component, derivative, processed form or purified form of any of the aforementioned.

In certain embodiments, the cell comprises a live cell.

In certain embodiments, the cell comprises a cell in vivo, an ex vivo cell, a cell in vitro, a cell in a biopsy and/or a cell in a biological fluid. The cell may be a live cell or a dead cell.

In certain embodiments, the method comprises intracellular imaging of the cell. In certain embodiments, the method comprises intracellular imaging of a live cell.

In certain embodiments, the method comprises obtaining one or more cells from a subject and exposing the cells so obtained to the complex. In certain embodiments, the method comprises exposing one or more cells obtained from a subject to the complex.

In certain embodiments, the method is used to screen for the presence or absence of a cancerous cell, or a cancer, in a subject.

In certain embodiments, the method is used to detect the presence or absence of a cancer in a subject.

In certain embodiments, increased labelling of the cell is indicative that the cell is a cancerous cell. In certain embodiments, the absence of increased labelling is indicative that the cell is a non-cancerous cell.

Certain embodiments of the present disclosure provide a method of identifying a cancerous cell in a subject, the method comprising exposing one or more cells from the subject to a complex comprising a transition metal carbonyl compound, a conjugated bidentate ligand and a tetrazolato compound to label the one or more cells and identifying a cancerous cell by increased labelling of the one or more cells by the complex.

Certain embodiments of the present disclosure provide a method of screening a subject for cancer, the method comprising exposing one or more cells from the subject to a complex comprising a transition metal carbonyl compound, a conjugated bidentate ligand and a tetrazolato compound to label the one or more cells and detecting the presence or absence of cancer in the subject on the basis of the labelling of the one or more cells by the complex.

Certain embodiments of the present disclosure provide a method of screening a subject for cancer, the method comprising exposing one or more cells from the subject to a complex comprising a transition metal carbonyl compound, a conjugated bidentate ligand and a tetrazolato compound to label the one or more cells and detecting the presence or absence of cancer in the subject on the basis of the labelling of the one or more cells by the complex, wherein an increased labelling of the one or more cells by the complex is indicative that the subject has cancer.

Certain embodiments of the present disclosure provide a method of detecting a disease, condition or state in a subject associated with altered or dysfunctional lipid intake, metabolism, processing, biogenesis or accumulation.

Certain embodiments of the present disclosure provide a method of detecting a disease, condition or state in a subject associated with altered or dysfunctional lipid intake, metabolism, processing, biogenesis or accumulation, the method comprising exposing one or more cells from the subject to a complex comprising a transition metal carbonyl compound, a conjugated bidentate ligand and a tetrazolato compound to label the one or more cells and detecting the disease, condition or state on the basis of the cells so labelled.

In certain embodiments, the method comprises intracellular imaging of the one or more cells. In certain embodiments, the method comprises intracellular imaging of one or more live cells.

In certain embodiments, the method comprises obtaining the one or more cells from a subject and exposing the cells so obtained to the complex. In certain embodiments, the method comprises exposing the one or more cells obtained from a subject to the complex.

In certain embodiments, the disease, condition or state comprises a cancer.

In certain embodiments, the one or more cells comprise one or more cancerous cells and the one or more cells have an increased labelling of the complex.

In certain embodiments, the increased labelling is as compared to a non-malignant cell.

In certain embodiments, increased labelling is indicative that the cell is a cancerous cell. In certain embodiments, the absence of increased labelling is indicative that the cell is a non-cancerous cell.

Certain embodiments of the present disclosure comprise a method of determining intracellular pH of a cell.

Certain embodiments of the present disclosure comprise a method of determining intracellular pH of a cell, the method comprising:
exposing the cell to a complex comprising a transition metal carbonyl compound, a conjugated bidentate ligand and a tetrazolato compound;
determining one or more emission characteristics from the complex in the cell; and
determining the intracellular pH on the basis of the one or more emission characteristics determined.

Examples of complexes are as described herein.

Examples of cells are as described herein. In certain embodiments, the cell comprises a live cell.

In certain embodiments, the intracellular pH comprises the pH of an intracellular structure. In certain embodiments, the intracellular structure comprises lipid.

In certain embodiments, the intracellular structure comprises a subcellular structure and/or a cell compartment.

In certain embodiments, the intracellular pH comprises the pH of a subcellular structure and/or a cell compartment. Examples of cell compartments include an acidic compartment, a lipid droplet and endoplasmic reticulum.

In certain embodiments, the subcellular structure and/or cell compartment comprises lipid.

Methods for detecting the complex in a cell are as described herein. Methods for determining the spectroscopic properties of the complexes, including the excitation and emission characteristics, are as described herein.

In certain embodiments, the one or more emission characteristics comprise emission intensity. For example, the intensity of emission at a wavelength that varies as a function of pH can be measured, such as 440 nm.

In certain embodiments, the one or more emission characteristics comprise the ratio of emission intensity at one wavelength to emission intensity at a second wavelength. For example, the intensity of emission at a wavelength that varies as a function of pH can be normalised against the intensity of emission at a wavelength that does not substantially vary as a function of pH, such as 560 nm.

In certain embodiments, the ratio of emission at 440 nm to 560 is used to determine pH.

Certain embodiments of the present disclosure provide an agent for measuring intracellular pH, the agent comprising a complex comprising a transition metal carbonyl compound, a conjugated bidentate ligand and a tetrazolato compound.

In certain embodiments, the agent is present in a solution of DMSO.

Certain embodiments of the present disclosure provide a composition for measuring intracellular pH, the composition comprising a complex comprising a transition metal carbonyl compound, a conjugated bidentate ligand and a tetrazolato compound.

In certain embodiments, the composition comprises DMSO. For example, a stock solution of the complex in DMSO may be prepared and the stock solution diluted in PBS for exposing to lipids, cells and cell structures.

The present disclosure is further described by the following examples. It is to be understood that the following description is for the purpose of describing particular embodiments only and is not intended to be limiting with respect to the above description.

### Example 1—Materials and Methods

Materials and Methods

(i) Fly Stocks

All stocks were maintained in standard medium at 25° C. The yeast GAL4-UAS system was used for targeted gene expression (Brand and Perrimon, 1993). Fat-body specific expression of transgenes from the UAS were driven by CG-GAL4 (Asha et al., 2003). Transgenic UAS-Atg9RNAi stocks (#34901) and UAS-TorTED stocks (#7013) were obtained from the Bloomington Drosophila Stock Centre (Indiana University, IN, USA). Atg8aGFP was driven by an endogenous promoter (Kelso et al., 2004).

(ii) Developmental Staging

Bromophenol-blue was added to the fly media and used to define different developmental time points. Third instar larvae at minus eight hours of puparium formation (−8PF) had a blue gut and were observed wandering in and out of the media, whereas at minus four hours (−4 h PF) the guts had less blue dye and the larvae were only observed out of the media. Newly formed white pupae (0 h PF), were isolated onto moist filter paper and incubated at 25° C. for either two hours (+2 h PF) or four hours (+4 h PF).

(iii) Nutrient Deprivation

Third instar larvae at −8 h PF were removed from standard media (normally feeding at this developmental stage) and placed on filter paper either soaked in 5% (w/v) sucrose (for amino acid deprivation), or 5% (w/v) tryptone pancreatic digest of casein (BD Bioscience USA; for carbohydrate deprivation), or soaked in water (for complete starvation). Larvae were incubated for 4-5 hours prior tissue isolation and imaging.

(iv) Tissue Isolation, Staining and Imaging

Fat body tissue was dissected into PBS from Drosophila larvae or pupae. The fat body tissue was either incubated with 10 μM tricarbonyl phenanthrolin (4-cyanophenyltretrazolato) Rhenium (I) in PBS for 15 minutes at room temperature, or LysoTracker® green (according to manufacturer instructions; Invitrogen, USA) and then mounted in carbomer-940 (Snowdrift farm, Tucson, USA) based optical coupling gel to prevent dehydration prior to imaging (Rothstein et al., 2006). For Oil Red O staining, fat body tissue was fixed in 4% (v/v) paraformaldehyde for 20 minutes, and incubated in 1/100 Oil Red 0 for 30 minutes and then washed in PBS before mounting in 80% glycerol.

A Ziess LSM710 META NLO confocal microscope (Zeiss, Germany), supplemented with a two-photon Mai-Tai® (tunable Ti:Sapphire femtosecond pulse laser, 710-920 nm, Spectra-Physics, Australia) was used for imaging. The images were acquired using a Plan-APOCHROMAT 63×/NA1.4 oil immersion objective. The Rhenium probe was excited at 830 nm using the two-photon pulse laser and detect at 600-654 nm. LysoTracker® green was imaged by excitation at 488 nm and detected at 497-558 nm. Atg8a-GFP was imaged with the Rhenium probe by excitation at 830 nm and resolved using spectral un-mixing using the Zen software package (based on spectral figure-printing of the two components). The Rhenium probe images and co-location analysis involved at least five separate images from five independent biological replicates, and one representative image from each treatment was selected for presentation (containing a minimum of 2 cells). For image quantification, two regions of interest (ROI) were selected at random with an area of 2500 μm² and the number of fluorescent puncta within the area counted, to give a minimum of 10 counts per treatment group. Comparison of means was performed by ANOVA with a Tukey post hoc tested in GraphPad Prism V.6.01 (USA).

(v) Rhenium Probe Binding of Lipids

To determine the binding properties of tricarbonyl-(4-cyanophenyltretrazolato)-phenanthroline-rhenium (I) (Rhenium probe) to various lipids, Rhenium probe-lipid overlay was performed using either lipids spotted onto PDVF membranes (Perkin Elmer, USA) or lipid MicroStrips (Echelon Biosciences Inc., Salt Lake City, Utah). The membranes contained at least 100 pmoles of each lipid in individual spots (supplementary table 1) or 10 μL (each in PBS) of 10% (v/v) glycerol, 1% (v/v) CHAPS, 1% (w/v) SDS, 1% (v/v) Tween20, Ecydsone (10 μg/100 μL), cholesterol (10 μg/100 μL), progesterone (10 μg/100 μL), PBS, or 1% (v/v) ethanol; and were incubated with 0.5 mM of Rhenium probe in 1% (w/v) BSA/PBS for 40 minutes and then washed four times for 10 minutes in PBS. Imaging was performed on an Image Quant Las 4000 (GE, Sweden) luminescent image analyser, with the Rhenium probe being detected by excitation at 460 nm using an epi-blue light source, and detected using an 605 nm ethidium bromide filter.

### Example 2—Rhenium Based Molecular Probe Detects Lipid Droplets in Drosophila Fat Body Tissue, but Relocates to an Acidic Compartment During Metamorphosis

A Rhenium based molecular probe, tricarbonyl phenanthrolin (4-cyanophenyltretrazolato) Rhenium (I), which reacts with and enables the imaging of lipid droplets, was used (Wright et al., 2012; Bader et al., 2014).

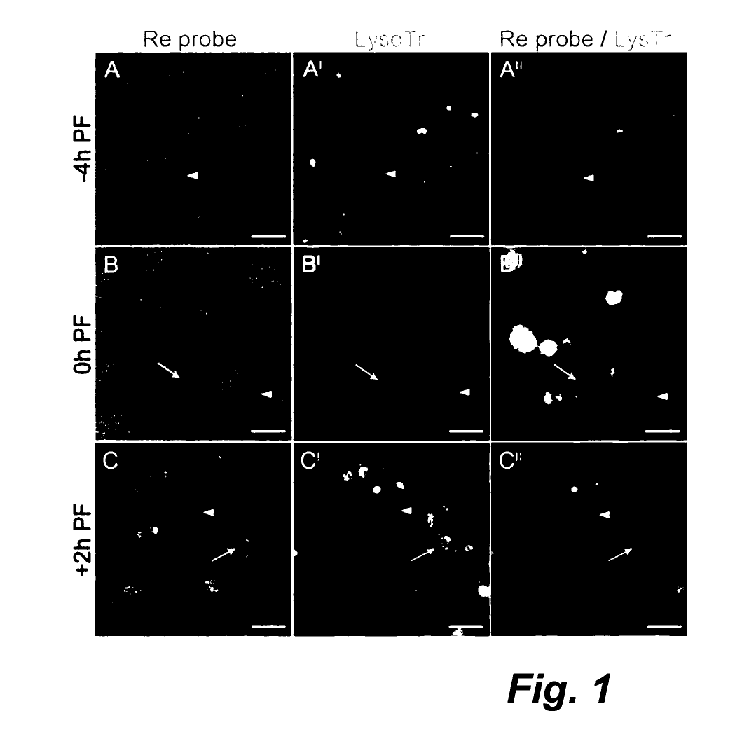

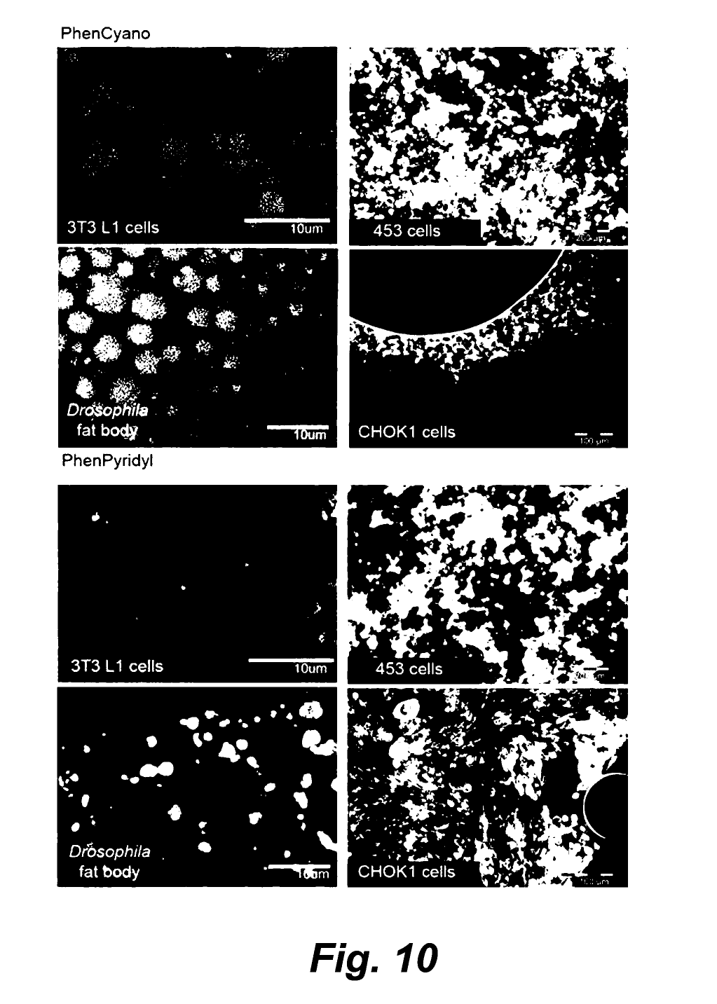

During Drosophila metamorphosis from larvae to adulthood, fat body tissue undergoes a lytic phase, during which lipid droplet storage is decreased, and energy stores are released to support the developmental process (Butterworth et al., 1988). To assess if the Rhenium probe could detect changes in lipid storage during Drosophila metamorphosis, fat body tissue was explanted at different developmental time points, including −4 h prior to puparium formation (PF), 0 h PF and +2 h PF. At −4 h PF the Rhenium probe interacted with the core of lipid droplets appearing to be spread evenly through the central matrix of these organelles (FIG. 1A, 1A^{II}). While at 0 h PF, most of the Rhenium probe was still inside lipid droplets (FIG. 1B, 1B^{II}), at +2 h PF there was a dramatic reduction in the amount of Rhenium probe interacting with lipid droplets (FIG. 1C, 1C^{II}). There was also a concomitant increase in the colocation of the Rhenium probe with acidic vesicles detected by LysoTracker® green, at +2 h PF (FIG. 1C^{I}, 1C^{II}), when compared to 0 h PF and −4 h PF (FIG. 1B^{I}, 1B^{II} and FIG. 1A^{I}, 1A^{II} respectively). Notably, the size of the lipid droplets did not appear to change dramatically at +2 h PF (FIG. 1C-1C^{II}), following the reduction in Rhenium probe staining, and compared to either 0 h PF (FIG. 1B-1B^{II}) or −4 h PF (FIG. 1A-1A^{II}). In contrast the size of the LysoTracker® green positive compartments increased at 0 h PF (FIG. 1B^{I}) and +2 h PF (FIG. 10, when compared to −4 h PF (FIG. 1A^{I}).

### Example 3—Rhenium Probe Reacts with Autophagosomes Following Starvation and the Induction of Developmental Autophagy

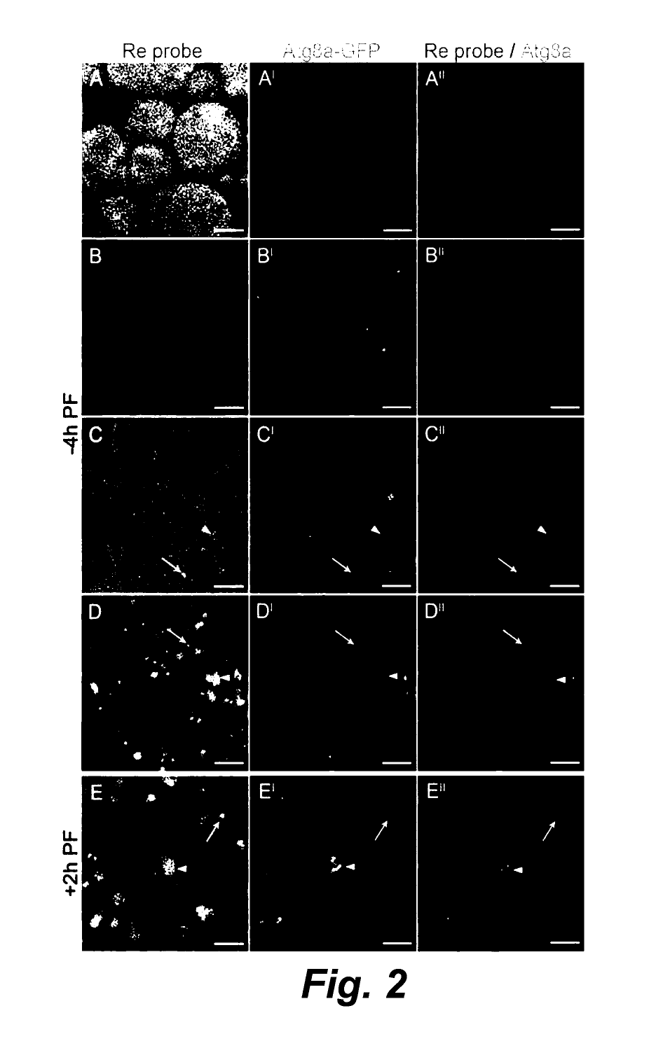

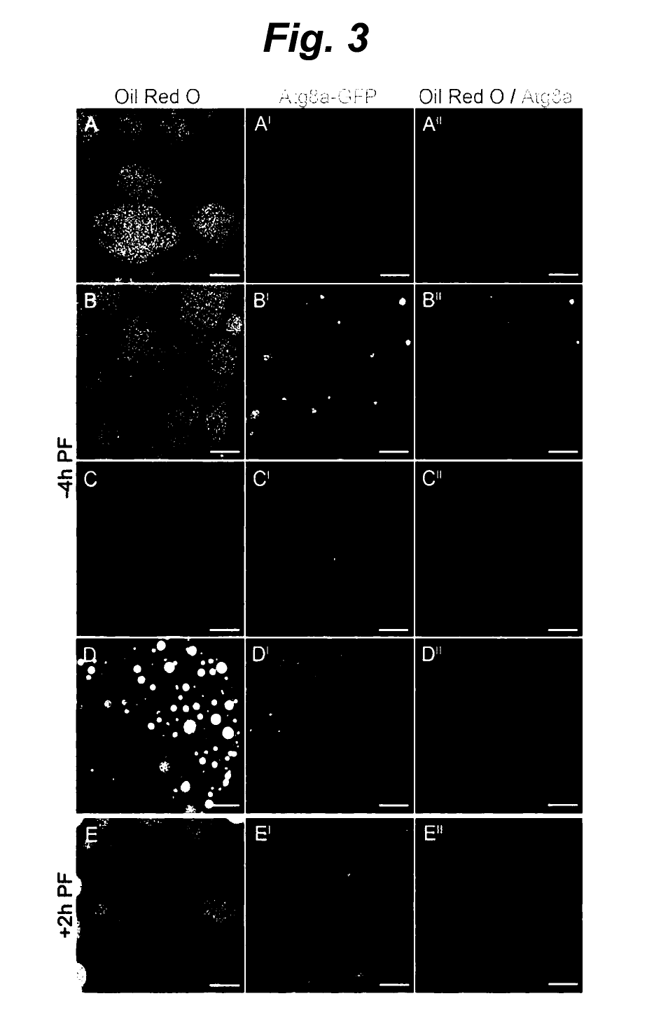

The increase in colocalisation of the Rhenium probe with Lysotracker® green positive acidic compartments at +2 h PF (FIG. 1C^{II}) coincides with increased autophagy at this developmental time point (Butterworth et al., 1988; Rusten et al., 2004). In addition, autolysosomes have been shown to have a role in lipid droplet degradation during pupal development (Singh et al., 2009a; Singh et al., 2009b). To confirm that the location of the Rhenium probe was changing from mainly lipid droplets at 0 h PF to autophagosomes at +2 h PF, the distribution of the Rhenium probe was defined in relation to the autophagosome marker Atg8a (ref). At −4 h PF there was minimal or no detectable Atg8a-GFP autophagic vesicles (FIG. 2A^{I}) and most of the Rhenium probe was detected in lipid droplets (FIG. 2A, 2A^{II}). However, at +2 h PF the Rhenium probe was detected mainly in association with Atg8a-GFP autophagosomes with only residual amounts of the probe being visualised in lipid droplets (FIG. 2E-E^{II}). Autophagy can also be induced by starvation and we hypothesised that this might also replicate this change in the Rhenium probe distribution from lipid droplets to autophagosomes during metamorphosis. At early developmental time points there is normally minimal autophagy, as evidenced here by the lack of Atg8a-GFP positive vesicles at −4 h PF (FIG. 2A^{I}). We examined the effect of starvation at this developmental time point and demonstrated that amino acid deprivation induced a small increase in punctate Atg8a-GFP autophagosomes (FIG. 2B^{I}), but no change in the distribution of the Rhenium probe (FIG. 2B), when compared to an untreated control (FIG. 2A). In contrast sugar deprivation induced more ATG8 GFP autophagosomes (FIG. 2C^{I}) and some change in the distribution of the Rhenium probe (FIG. 2C, 2C^{II}). Furthermore, starvation (amino acid and sugar deprivation), induced a dramatic redistribution of the Rhenium probe to Atg8a-GFP autophagosomes and resulted in both reduced size and Rhenium probe staining of lipid droplets (FIG. 2D^{I-II}). Under these starvation conditions approximately 90±11% of the vesicles staining with the Rhenium probe were also Atg8a-GFP positive. The pattern of neutral lipid staining detected by oil red was quite distinct from the Rhenium probe under conditions of both starvation and developmental autophagy (FIG. 3 and FIG. 2 respectively). In particular the pattern of oil red staining was not markedly changed during developmental autophagy (FIG. 3E, 3E^{II}), whereas the Rhenium probe distribution was markedly changed (e.g. FIG. 1E^{II}) when compared to earlier developmental time points (e.g. FIG. 1A^{II}). In addition, the oil red staining did not appear to colocalise with Atg8a-GFP under any of the nutrient deprivation/starvation conditions (FIG. 3), despite for example, a reduction in lipid droplet size following starvation (amino acid and sugar deprivation; FIG. 3D^{II}). This indicated that the Rhenium probe staining was quite distinct from oil red staining and that the probe was most likely not interacting with neutral lipids.

### Example 4—Constitutive Tor^{TED} Activation of Autophagy and Atg9 Silencing Alter Rhenium Probe Distribution

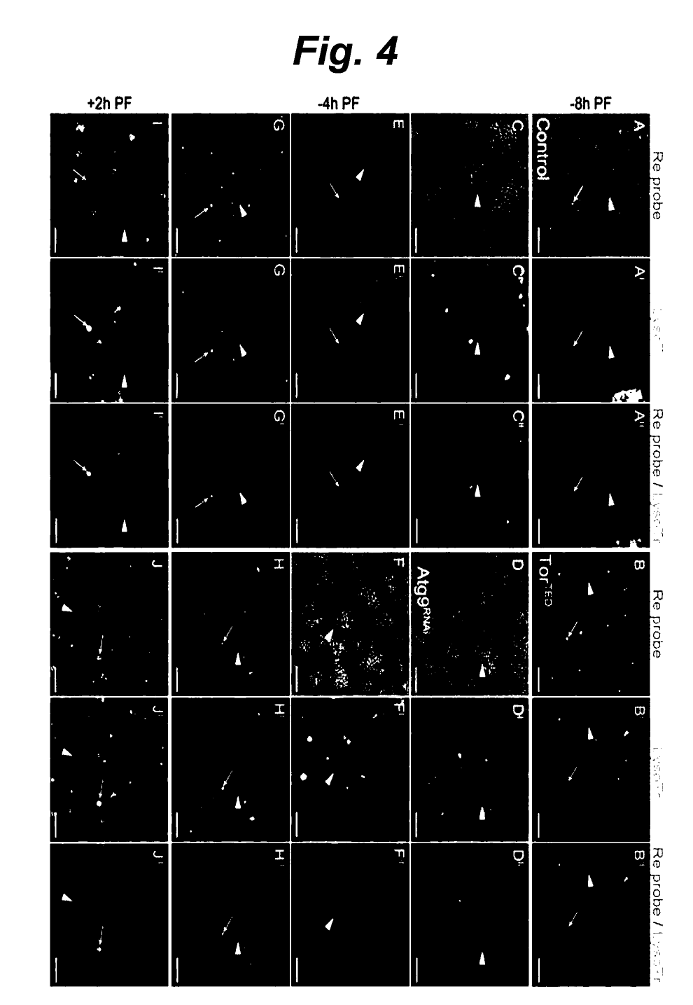

Next we assessed how the genetic modulation of autophagy affects the Rhenium probe distribution in lipid droplets and autophagosomes. The target of rapamycin (Tor) is a negative regulator of autophagy (Scott et al., 2004, Noda and Ohsumi, 1998), receiving inputs from intracellular and extracellular stimuli that include nutrients and growth factors. At −8 h PF when larvae are normally feeding and there is no autophagy, the expression of dominant negative TorTED resulted in almost all of the Rhenium probe in LysoTracker® green positive vesicles, with little or no lipid droplet staining (FIG. 4B-4B^{II}), when compared to controls (FIG. 4A-4A^{II}).

The silencing of Atg9 (Atg9^{RNAi}) has been shown to decrease the normal autophagic response to starvation in larval fat body tissues at −4 h PF (Low et al., 2013, Tang et al., 2013, Nagy et al., 2014). Under normal feeding conditions at −4 h PF larvae expressing Atg9^{RNAi} showed a small amount of punctate LysoTracker® green staining that was distinct from the Rhenium probe, and which was detected mainly in lipid droplets (FIG. 4D-4D^{II}), with a similar distribution to that in controls (FIG. 4C-4C^{II}). In Atg9^{RNAi} larvae at −4 h PF, amino acid deprivation had little or no effect on the distribution of the Rhenium probe in lipid droplets (FIG. 4F-4F^{II}), whereas controls showed some reduction in lipid droplet staining and a few LysoTracker® green vesicles were collocating with the Rhenium probe. In contrast, Atg9^{RNAi} larvae at −4 h PF, starvation (-amino acids and -sugar; FIG. 4H-H^{II}) resulted in Rhenium probe distribution that was mainly in LysoTracker® green vesicles, with little or no lipid droplet staining; which was similar to the controls at this developmental time point (FIG. 4G-G^{II}). In Atg9^{RNAi} larvae at +2 h PF (normally exhibiting developmental autophagy at this time point) almost all of the Rhenium probe was detected in small LysoTracker® green vesicles (FIG. 4J-J^{II}), but while the +2 h PF control had similar vesicles there was still some lipid droplet staining and larger LysoTracker® green vesicles (FIG. 4^{I-III}).

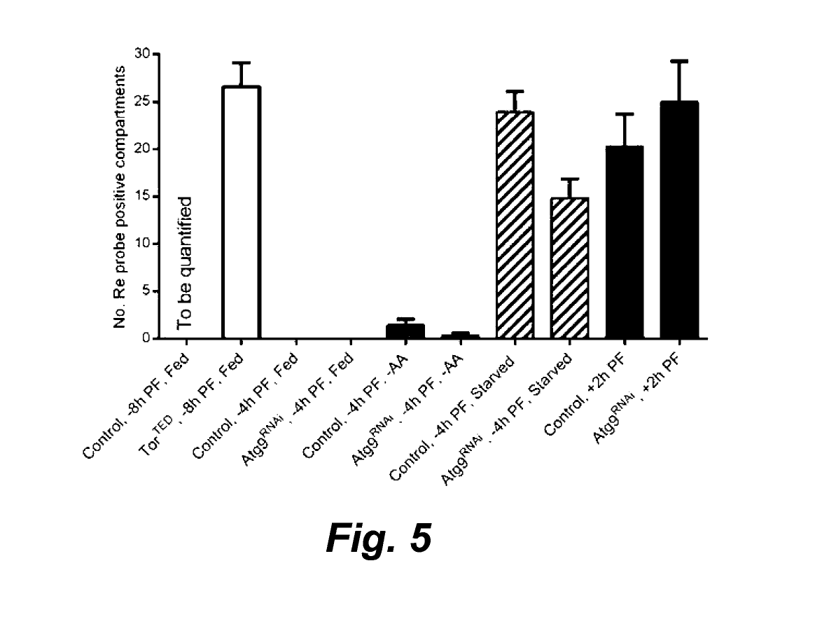

Consequently, the number of Rhenium and LysoTracker® green positive compartments were increased significantly (FIG. 5) under either starvation (either with or without Atg9^{RNAi}) developmental autophagy (+2 h PF, either with or without Atg9^{RNAi}) and with constitutive activation of autophagy (Tor^{TED}).

### Example 5—Rhenium Probe Interacts with Steroid Lipids

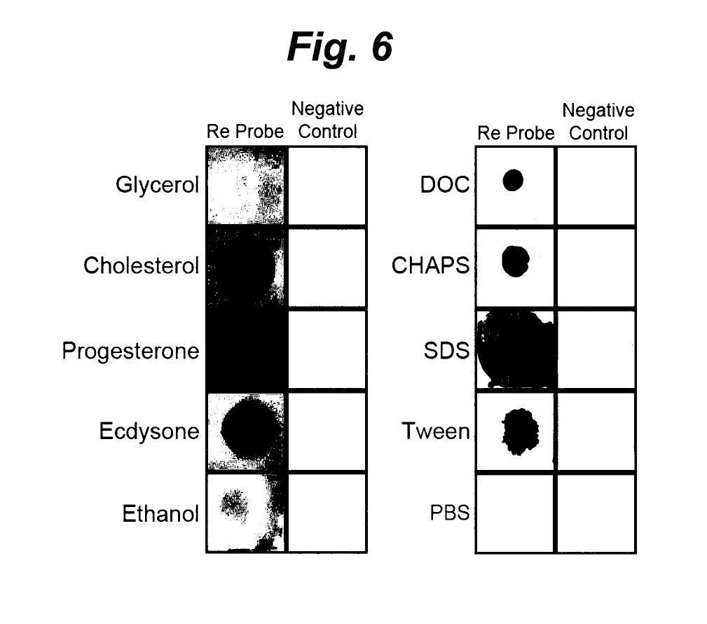

FIG. 6 shows that Rhenium molecular probe interacted with lipids and detergents. The figure shows a Rhenium molecular probe-lipid overlay, showing an interaction between the Rhenium molecular probe (Re Probe) and three lipid species (cholesterol, progesterone and edysone) and four detergents (DOC, CHAPS, SDS, Tween) and no interaction with controls (glycerol, ethanol) or cell media (PBS). Neither albumin, transferrin (serum proteins that have been identified in lipid droplets isolated from mouse mammary glands), larval serum protein 1 (expressed in high levels in larval lipid droplets, but not adult flies), nor perilipin (human plasma protein that has been detected in lipid droplets) reacted with the Rhenium probe. Similarly neutral lipids and long chain fatty acids did not react with the Rhenium probe, while some residual background reactivity was evident with two detergents sodium dodecyl sulphate (SDS) and Tween® 20 (i.e. auto-reactivity detected at 575-605 nm with SDS and Tween® 20 only on PDF membranes). In contrast, the Rhenium probe demonstrated a high level of reactivity with both deoxycholate and CHAPS and also interacted with cholesterol, progesterone and the Drosophila hormone ecdysone (FIG. 6).

Discussion: Lipid droplets are utilised by cells to store excess lipids, which are usually sequestered as triglycerides and cholesterol esters. Lipid droplets act as a critical energy source for cells during high demand or nutrient deprivation and also isolate potentially toxic lipids from other metabolic processes. Autophagosomes have a pivotal role in lipid localisation, orchestrating the vesicular trafficking and secretion of triglyceride rich lipoproteins and transient storage of lipids in lipid droplets. Autophagosomes also appear to recruit lipids for membrane expansion and have a specific role in lipid degradation and fuelling the energy generating pathways of (3-oxidation in mitochondria. The dynamic balance between lipid droplets and autophagosomes therefore has a fundamental role in lipid homeostasis, energy metabolism and cellular function.

Lipid droplets are thought to initially form by triglyceride exclusion from microsomal membranes, giving rise to constituents that include, the microsomal associated membrane protein caveolin, the heat shock protein GRP78, adipocyte differentiation protein, vimentin and the extracellular signal-regulated kinase-2 (ERK2) responsive phospholipase D. A number of methods have been developed to detect lipid droplets in cells, and these rely mainly on either dyes or molecular probes that interact with these molecular constituents or the core lipids in these vital organelles. The Rhenium(I) tricarbonyl phenanthroline complex that was investigated here interacted with a molecular constituent of lipid droplets that was evenly distributed throughout the matrix of the lipid droplets, and the staining appeared to be granular, and suggestive of sub-compartmentalisation. Unlike Nile Red and Oil Red 0 dyes, the Rhenium complex did not require cell/tissue fixation, enabling live cell imaging, which will have applications for critical studies on lipid droplet biology and function.

The dynamic relationship that is emerging between lipid droplets and autophagy appears to be central in key cell biological processes like energy sensing and metabolism. The fundamental interactions that have already been elucidated include: the transfer of membrane constituents to facilitate autophagosome formation and biogenesis; cholesterol and cholesterol ester transfer and modification, which is important in membrane structure and fluidity; neutral lipid transfer to lipid droplets for storage, or back to autophagosomes for degradation and incorporation into energy/metabolic pathways; complete lipid droplet organelle autophagocytosis, presumably during stress or high energy demand or organelle turnover. The Rhenium tetrazolato complex acted as a molecular probe that was transferred from lipid droplets to autophagosomes, and this was evident at specific developmental time-points and other conditions that induced autophagy. There was a significant transition of the Rhenium molecular probe from the core matrix of lipid droplets in Drosophila fat body tissue to acidified amphisomes-autolysosomes at the developmental stage of four hours prior to puparium formation. However, in response to either combined glucose and amino acid starvation or the inactivation of the target of rapamycin (TorTED activates the autophagy cascade through phosphorylation of Atg13) there was almost complete transfer of the Rhenium molecular probe from lipid droplets to acidified amphisome-autolysosomes. This indicated that the Rhenium molecular probe might be a reporter for a specific event during the interaction of lipid droplets and autophagosomes.

The molecular target for the Rhenium molecular did not appear to be detecting the induction of autophagy as under conditions of amino acid deprivation autophagosomes were formed, but there was little or no molecular probe interaction with these autophagosomes. The data also suggest that the molecular probe was not tracking at least the initial stages of autophagosome membrane formation and PNPLA5 mediated early biogenesis of autophagosomes. The Rhenium molecular probe demonstrated strong interaction with either detergents containing a steroid group or cholesterol and its steroid hormone derivatives, including Drosophila ecdysone and human progesterone. Esterified cholesterol that is stored in lipid droplets can be transferred to autophagosomes and undergoes lipase hydrolysis to form cholesterol, which presumably occurs in amphisomes as cholesterol depletion impedes autophagosome maturation and the formation of functional autolysosomes. This could be consistent with the Rhenium molecular probe detecting cholesterol that was transferred from lipid droplets to autophagosomes during this maturation process. Thus, we observed significant amounts of molecular probe in acidified autolysosomes produced in response to maximum stimulation of autophagy by TorTED or combined amino acid and glucose starvation.

### Example 6—Use of Probes

The probes described herein may be used, for example, to assist with diagnosis of various conditions in which fat may appear in abnormal locations.

For instance, bone fractures or crush injuries to fatty regions of the body may release fat into the bloodstream, leading to fat emboli which can be fatal—these emboli may be detected using the probes as described herein. They may also be useful to identify tumors, such as lipomas and liposarcomas, which arise from fat cells. Deposits of fat may also appear in the liver and kidney in a variety of pathological conditions, and these may be detected using the probes as described herein.

For example, a biopsy from a tissue of interest may be obtained by a standard procedure. The tissue biopsy may be processed by dissection into PBS. The tissue may then be incubated with 10 μM tricarbonyl phenanthrolin (4-cyanophenyltretrazolato) Rhenium (I) in PBS for 15 minutes at room temperature and then mounted in carbomer-940 (Snowdrift farm, Tucson, USA) based optical coupling gel to prevent dehydration prior to imaging.

Alternatively, the tissue sample may be fixed in 4% (v/v) paraformaldehyde for 20 minutes, and then incubated with a solution of 10 μM tricarbonyl phenanthrolin (4-cyanophenyltretrazolato) Rhenium (I) in PBS for 30 minutes and then washed in PBS before mounting in 80% glycerol, and visualized.

Amongst other uses contemplated, the use of the probes as described herein for cholesterol testing or for detecting steroids in athletes is also contemplated.

### Example 7—Rhenium Probes

The Rhenium probes used are shown in Table 1.

TABLE 1

| Abbreviation | Compound | Dye structure |
|---|---|---|
| PhenCyano | C₂₃H₁₂N₇O₃Re Triscarbonyl phenanthroline (4- cyanophenyltetrazolato)Rhenium(I) Alternatively: facial-Tricarbonyl (η¹(N2)-5-cyanophen-4′-yl tetrazolato) η²-1,10-phenathroline Rhenium(I) | 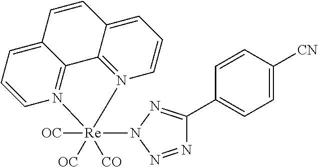 |
| PhenPyridyl | Triscarbonyl phenanthroline (3- pyridyltetrazolato)Rhenium(I) C₂₁H₁₂N₇O₃Re Alternatively: facial-Tricarbonyl (η¹(N2)-5-pyrid-3′-yl tetrazolato) η²- 1,10-phenathroline Rhenium(I) | 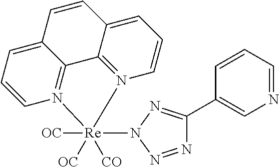 |
| PhenEster | C₂₄H₁₄N₆O₅Re facial-Tricarbonyl (η¹(N2)-5-(methyl benzoate-4′-yl tetrazolato) η²-1,10- phenathroline Rhenium(I) | 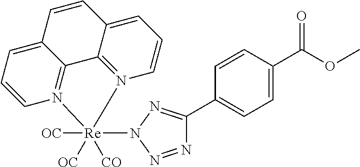 |
| BiphenCyano | C₂₁H₁₂N₇O₃Re facial-Tricarbonyl (η¹(N2)-5- cyanophen-4′-yl tetrazolato) η²-2,2′- bipyridine Rhenium(I) | 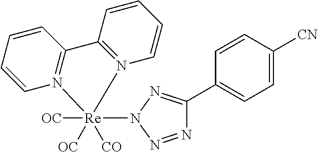 |
| BiphenEster | C₂₂H₁₄N₆O₅Re facial-Tricarbonyl (η¹(N2)-5-(methyl bcnzoate-4′-yl tetrazolato) η²-2,2′- bipyridine Rhenium(I) | 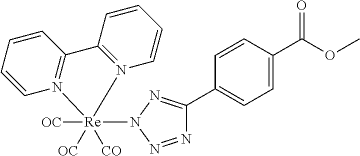 |

### Example 8—Rhenium Molecular Probes Lipid Interaction

Evidence of Molecular Probe Interaction with Lipids

The experimental protocol probe-lipid overlay was as follows: 50 μM of each lipids was loaded on to PVDF membranes (Perkin Elmer, USA), which were then incubated with molecular probes. PhenCyano and PhenPyridyl were assessed against eleven lipids including; sphingomyelin (Cat # S0756, Sigma Aldrich, USA), sphingosine (Cat # S7049; Sigma Aldrich, USA), L-phosphatidylethanolamine (Cat # P7943, Sigma Aldrich, USA), monosialodanglioside (GM1; Cat # G7641, Sigma Aldrich, USA), oleoly-L-?-lysophosphatidic acid (Cat # L7260, Sigma Aldrich, USA), palmitic acid (Cat # P5917, Sigma Aldrich, USA), sphingomyelin (Cat # S0756, Sigma Aldrich, USA), sphingosine (Cat # S7049; Sigma Aldrich, USA), L-?-phosphatidylethanolamine (Cat # P7943, Sigma Aldrich, USA), monosialodanglioside (GM1; Cat # G7641, Sigma Aldrich, USA), progesterone (Cat #102722, MP Biomedical, USA), ceramide (Cat # C2137; Sigma Aldrich, USA), triacylglycerol mix C2-C10 (Cat #17810, Sigma Aldrich, USA) and cholesteryl acetate (Cat #151114; Sigma Aldrich, USA).

All other molecular probes were assessed against a panel of six lipids including; sphingomyelin (Cat # S0756, Sigma Aldrich, USA), sphingosine (Cat # S7049; Sigma Aldrich, USA), L-?-phosphatidylethanolamine (Cat # P7943, Sigma Aldrich, USA), monosialodanglioside (GM1; Cat # G7641, Sigma Aldrich, USA), sphingomyelin (Cat # S0756, Sigma Aldrich, USA), sphingosine (Cat # S7049; Sigma Aldrich, USA), L-?-phosphatidylethanolamine (Cat # P7943, Sigma Aldrich, USA), monosialodanglioside (GM1; Cat # G7641, Sigma Aldrich, USA) and triacylglycerol mix C2-C10 (Cat #17810, Sigma Aldrich, USA).

Lipids were dissolved in ethanol, methanol or isopropanol for loading depending on solubility. Following lipid loading, membranes were left to dry for 20-30 minutes, then they were washed for 10 minutes in cold 10% ethanol, before they were incubated with 10 μM solution of each Rhenium molecular probe in cold 10% ethanol for one hour. Membranes were than washed four times for 10 minutes in cold 10% ethanol prior to imaging. Imaging was performed on an Image Quant Las 4000 (GE, Sweden) luminescent image analyser, with the Rhenium molecular probe being detected by excitation at 460 nm using an epi-blue light source, and detected using an 575 nm ethidium bromide filter.

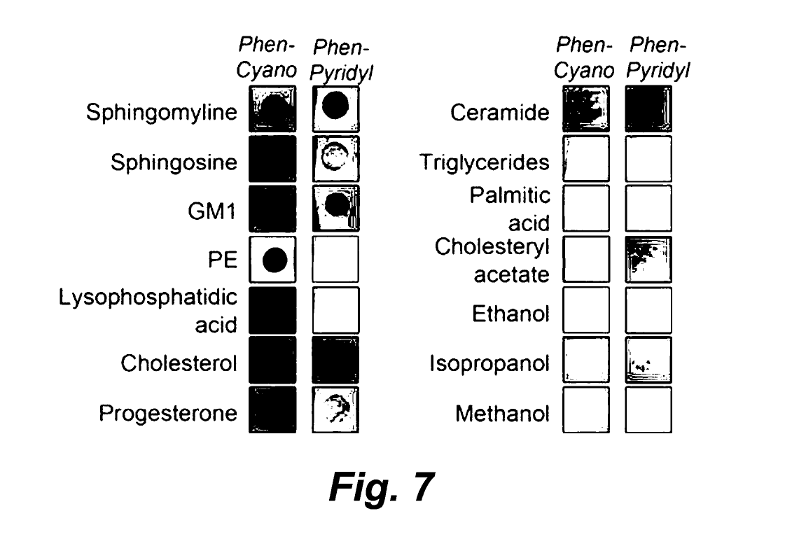

The results for PhenCyano and PhenPyridyl probes are shown in FIG. 7, which shows a Rhenium molecular probe-lipid overlay, demonstrating an interaction between the probe and seven lipid species on the left and no interaction with four lipid species on the right or controls (ethanol, isopropanol or methanol). Blank shows detection of lipid background, without Rhenium molecular probe incubation. PhenCyano and PhenPyridyl showed some affinity towards polar lipids, sphingomyelin, sphingosine, monosialotetrahexosylganglioside (GM1), phosphatidylethanolamine (PE), lysophosphatidic acid, cholesterol and progesterone. There was no interaction detected with neutral lipids, ceramide, triglycerides, fatty acid (palmitic acid), cholesterol ester (cholesteryl acetate), or with controls ethanol, isopropanol or methanol.

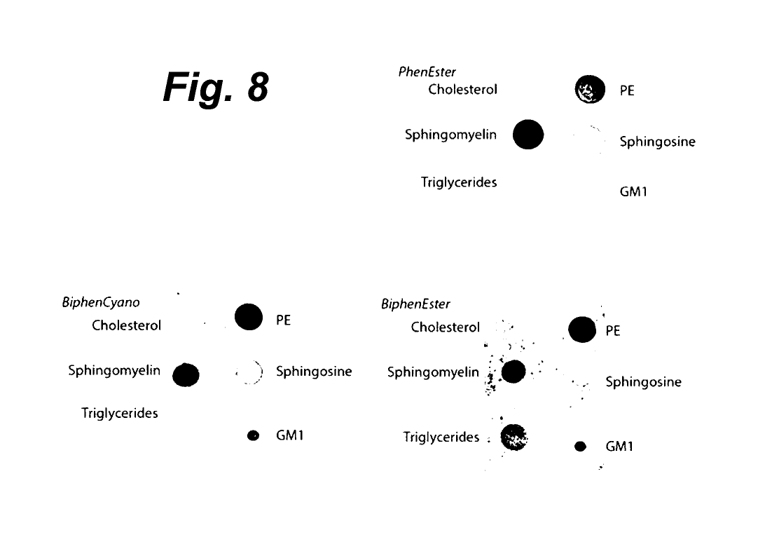

The results for the PhenEster, BiphenEster, BiphenCyano probes are shown in FIG. 8, which provides a rhenium molecular probe-lipid overlay, showing varying interactions between the Rhenium molecular probes as labelled (Re Probe) and six lipid species.

PhenEster, BiphenEster, BiphenCyano all showed varying affinity towards polar lipids, cholesterol, phosphatidylethanolamine (PE), sphingomyelin, sphingosine and monosialotetrahexosylganglioside (GM1). PhenEster and BiphenEster also appeared to interact with neutral triglycerides, but the others did not. The results are provided in Table 2.

TABLE 2

| | Phen | Phen | Phen | Biphen | Biphen |
|---|---|---|---|---|---|
| | Cyano | Pyridyl | Ester | Cyano | Ester |
| Sphingomyline | ✓ | ✓ | ✓ | ✓ | ✓ |
| Sphingosine | ✓ | X | ✓ | ✓ | X |
| GM1 | ✓ | ✓ | ✓ | ✓ | ✓ |
| PE | ✓ | ✓ | ✓ | ✓ | ✓ |
| Lysophosphatidic | ✓ | ✓ | n/a | n/a | n/a |
| acid | | | | | |
| Cholestrol | ✓ | X | ✓ | ✓ | X |
| Progesterone | ✓ | X | n/a | n/a | n/a |
| Ceramide | x | X | n/a | n/a | n/a |
| Triglycerides | X | X | ✓ | x | ✓ |
| Palmitic acid | X | X | n/a | n/a | n/a/ |
| Cholestryl | X | x | n/a | n/a | n/a |
| acetate | | | | | |

Tabulated results for interactions between named probes and named lipids from dot blot results; ✓ indicates a positive interaction, x indicates no interaction and n/a indicates this specific interaction was not tested for.

These results demonstrate that the interactions of the probes are dependent upon the probe structure.

### Example 9—Cell Staining with Rhenium Molecular Probes

Staining Protocol for Rhenium Molecular Probes

(i) Stock solution of Probes

Probes were dissolved in DMSO to obtain a stock solution with a concentration of 10 mM. Stock solutions were stored at room temperature in a dark place. Working solutions were prepared only at the time of use as the probe has reduced solubility in aqueous solutions. Working calculations for each of the probes used are shown in Table 3. A Working solution was a dilution 1/1000 to 1/500 of the stock in DMSO to PBS or cell medium, and as such the percentage of DMSO used in actual cell experiments was less than 1%.

TABLE 3

| | | Weight of | Volume |
|---|---|---|---|
| | Molecular | Compound | DMSO |
| Dye | Mass | (mg) | (μL) |
| PhenCyano | 620.59 | 3.10295 | 500 |
| PhenPyridyl | 596.57 | 2.98285 | 500 |
| PhenEster | 653.62 | 3.2681 | 500 |
| BiphenCyano | 596.57 | 2.98285 | 500 |
| BiphenEster | 629.6 | 3.148 | 500 |

(ii) Staining of Live Samples

For tissues, the tissues were isolated in PBS (or other physiological media) and mounted on a coverslip. For cells, these were grown as per normal practice on coverslips.

The media was removed media and replaced with 10-20 μM solution of molecular probes in PBS (or appropriate physiological media/cell media) at a dilution of stock solution from 1/1000-1/500, for 15-30 minutes at physiologically appropriate temperature (37° C. for cell culture or 25° C. for insect larvae). In this regard, it was found that it was preferable that the media does not contain foetal calf serum, or other high lipid content ingredients. If foetal calf serum is necessary for cell health, an increased incubation time may be necessary to obtain adequate staining.

The samples were washed for one minute in PBS. If co-staining was performed, the samples were washed in PBS for 30 seconds, before incubating with counterstain.

Tissues were mounted in optical coupling gel, such as carbomer-940 (Snowdrift farm, Tucson, USA) based gel to prevent dehydration prior to imaging and maintain tissue integrate (Rothstein, E. C., Nauman, M., Chesnick S., Balaban R. S., 2006, Multi-photon excitation microscopy in intact animals Journal of Microscopy 222, 58, 64). For cells, the coverslip was mounted in PBS for immediate imaging.

Samples were imaged immediately following staining.

Staining of Fixed Samples

Following fixation (paraffin embedding or alcohol based fixation) samples were washed three times for five minutes in PBS at room temperature.

Samples were incubated with 10-20 μM solution of the molecular probe in PBS (1/1000-1/500 dilution of stock) for 20-30 minutes for alcohol or paraformaldehyde fixed samples, or 40 minutes to one hour for paraffin embedded tissue sections at room temperature, with agitation.

Samples were washed three times for five minutes in PBS at room temperature, with agitation and mounted in 80% glycerol for imaging. Samples can be stored overnight at room temperature in a dark cupboard.

(iii) Imaging Rhenium Molecular Probes

(a) Epi-Fluorescence Microscopy

Rhenium molecular probes were excited by a UV or blue light sources (eg 405 nm). Image collection was performed with a wideband pass filter within the range of 500-650 nm, or narrowband pass filter within this emission range. Photobleaching may occur with mercury light sources if multiple colour imaging is being performed, i.e. if the sample is excited at multiple wavelengths at once.

(b) Two-Photon and Confocal Microscopy

Rhenium molecular probes were? excited at 800-830 nm using a two-photon pulse laser or 405 steady state laser and detection in the range of 490-670 nm, with an emission maxima at around 570.

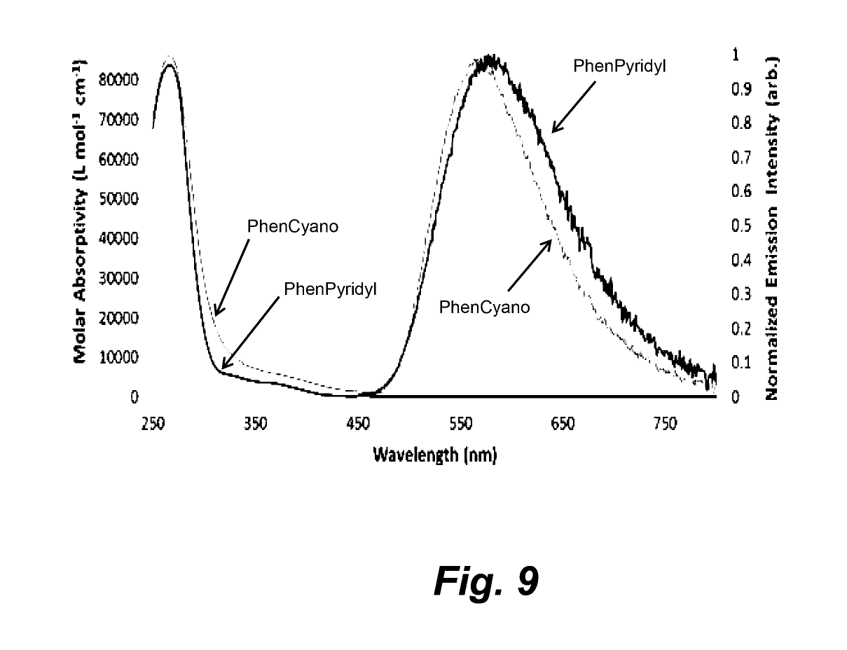

The excitation-emission profile for the rhenium molecular probes, PhenCyano and PhenPyridyl obtained is shown in FIG. 9. The figure shows absorption and emission profiles of complexes 1 and 2 from a diluted (ca. 10⁻⁵ M) air-equilibrated H₂O/DMSO 99:1 solution at room temperature.

### Example 10—Samples Stained with Rhenium Molecular Probes

Micrographs of 3T3 L1, 453 cells and CHOK1 cells and Drosophila fat body tissue stained with rhenium molecular probes, PhenCyan top, PhenPyridyl bottom are shown in FIG. 10. The figure shows images collected using two-photon microscopy of the PhenCyano and PhenPyridyl probes incubated with live samples, as indicated in the image. Left: confocal images; Right: fluorescence microscopy images.

The PhenCyano probe displayed a distribution consistent with lipid droplet staining in 3T3 L1 cells and Drosophila fat body cells. The PhenPyridyl probe showed an interaction with smaller vesicular structures that were more dispersed within the cells and there was limited to no lipid droplet staining in either 3T3 L1 cells or Drosophila fat body cells. The pattern of detection for the PhenPyridyl probe in Drosophila fat body cells was more indicative of acidic compartments and the staining intensity was increased. Both molecular probes gave similar staining patterns in 453 and CHO-K1 cells, which again had a higher intensity and was consistent with the detection of acidic compartments. These findings were consistent with the PhenCyano probe detecting lipids within lipid droplets and autophagosomes/acidic compartments whereas the Phenpyridyl compound only appeared to detect lipids within autophagosomes/acidic compartments.

### Example 11—Micrographs of Murine Brain from Control and Lysosome Storage Disorder Model MPS IIIA Mice Stained with PhenCyano Rhenium Molecular Probe

Mouse brains from control and lysosome storage disorder model MPS IIIA mice (Gliddon B L and Hopwood J J (2004) “Enzyme-Replacement Therapy from Birth Delays the Development of Behavior and Learning Problems in Mucopolysaccharidosis Type IIIA Mice” Pediatric Research 56, 65-72) were obtained and stained with PhenCyano Rhenium molecular probe as described above, paraffin embedded and sectioned at 6 μm prior to staining.

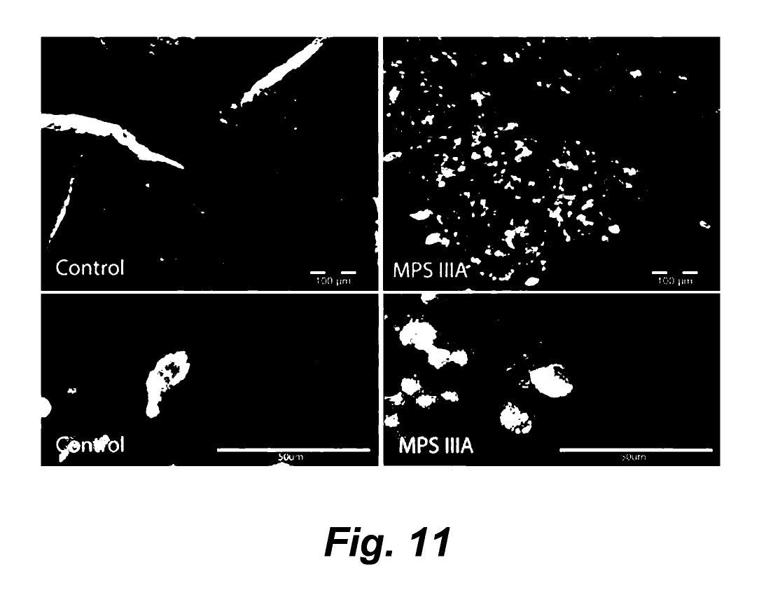

The results are shown in FIG. 11.

The results demonstrate that the probes have potential as diagnostic tools for pathologies which have lipid accumulation associated with them. For example lysosome storage disorders, where lipids accumulate in lysosomal storage compartments in tissues including the brain, leading to neuronal defects. The staining pattern of FIG. 11 for control neurons is consistent with normal neuronal architecture and lipid distribution, whereas the lysosomal storage disorder micrograph shows altered neuronal architecture and evidence of lipid storage within neuronal cells.

### Example 12—PhenCyano Rhenium Molecular Probe Detects Lipid Specific Autophagy

Autophagy is a cellular recycling process which allows the degradation of cytoplasmic content in response to specific stress conditions. Under some stress conditions autophagy targets lipids stored in lipid droplets for degradation to provide cells with extra energy.

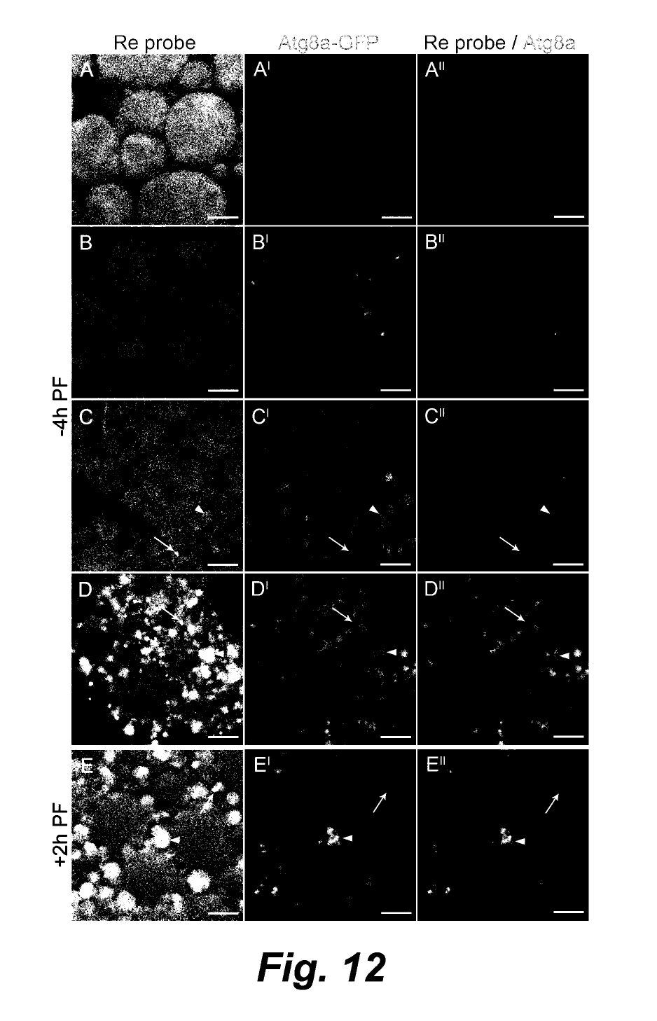

FIG. 12 shows confocal micrographs of Drosophila fat body tissue from larvae at −4 h PF (A-D^{II}) or pupae at +2 h PF (E-E^{II}), stained with Rhenium molecular probe PhenCyano (grey scale in A, B, C, D and E; red in A^{II}, B^{II}, C^{II}, D^{II} and E^{II};) and expressing Atg8a-GFP (grey scale in A^{I}, B^{I}, C^{I}, D^{I} and E^{I}; green in A^{II}, B^{II}, C^{II}, D^{II} and E^{II}). Tissue from larvae at −4 h PF on standard feeding medium (A-A^{II}), −4 h PF larvae deprived of amino acids for 4 h (B-B^{II}), −4 h PF larvae deprived of sugar for 4 h (C-C^{II}), −4 h PF larvae starved for 4 h (deprived of amino acids and sugar; D-D^{II}) and pupae at +2 h PF (E-E^{II}). Arrowheads indicate Rhenium molecular probe collocating with Atg8aGFP positive compartments (C-E^{II}), arrows indicate Rhenium molecular probe alone (C-E^{II}). Scale bars=10 μm. The results demonstrate that the rhenium molecular probe localised with autophagosomes under conditions which induce lipid specific autophagy.

Conditions which induced cells of the Drosophila fat body tissue to begin degrading lipid droplets via autophagy also induced co-localisation between the PhenCyano Rhenium molecular probe and autophagy marker Atg8a-GFP. Thus we were able to detect autophagic compartments which contained lipids using this PhenCyano Rhenium molecular probe.

The results demonstrate that the rhenium molecular probe localised with autophagosomes under conditions which induce lipid specific autophagy.

Conditions which induced cells of the Drosophila fat body tissue to begin degrading lipid droplets via autophagy also induced co-localisation between the PhenCyano Rhenium molecular probe and autophagy marker Atg8a-GFP. Thus we were able to detect autophagic compartments which contained lipids using this PhenCyano Rhenium molecular probe.

### Example 13—PhenCyano Rhenium Molecular Probe as a pH Biosensor in Live Cells

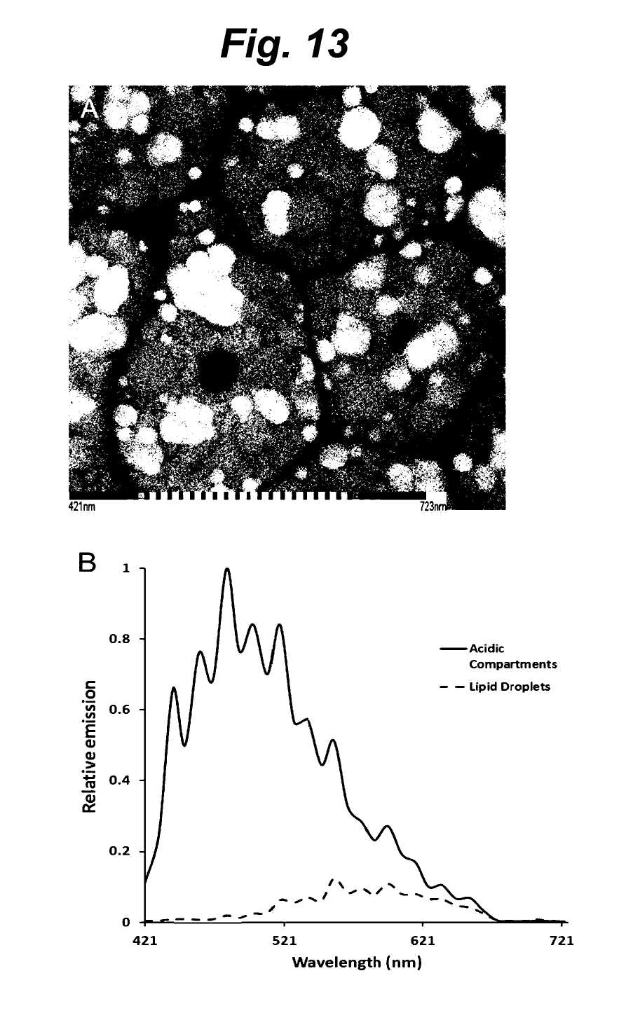

FIG. 13 shows in panel (A) confocal micrographs of Drosophila fat body at +2 h PF stained with PhenCyano rhenium molecular probe showing spectral profile of probe in acidic compartments (cyan) and lipid droplets (green/yellow). Panel (B) shows the spectral emissions profile of the rhenium molecular probe in different subcellular compartments (acidic compartments=solid line: lipid droplets=dashed line) relative to emission maxima of acidic compartment (intensity at λₑₘ 480 nm). The results show that the emission spectral profile of the PhenCyano rhenium molecular probe in the acidic compartment following autophagic inducement was found to have undergone a blue shift and an increase in emission intensity. This change can be related to the change in cellular pH within the acidic compartment compared with the lipid droplet (location of probe prior to autophagic induction).

### Example 14—The PhenCyano Rhenium Molecular Probe is a pH Sensor

Experimental conditions CaryEclipse Fluoriometer. The fluorescence emission of the PhenCyano rhenium molecular probe was measured as a function of pH using a Varian CaryEclipse Fluoriometer. spectrophotometer at room temperature. All pH measurements were conducted using an Orion Ross pH meter. Optical spectroscopy experiments were recorded in 50:50 water:methanol at constant ionic strength (I=0.01 (NaCl)) Deionised water; that had been purified with the MiliQ-Reagent system to produce water with a specific resistance of >18.2 M S2 cm⁻¹, then boiled for 30 min to remove CO₂ and cooled under a drying tube filled with soda lime; was used to prepare all aqueous solutions. All solutions were prepared freshly prior to measurement.

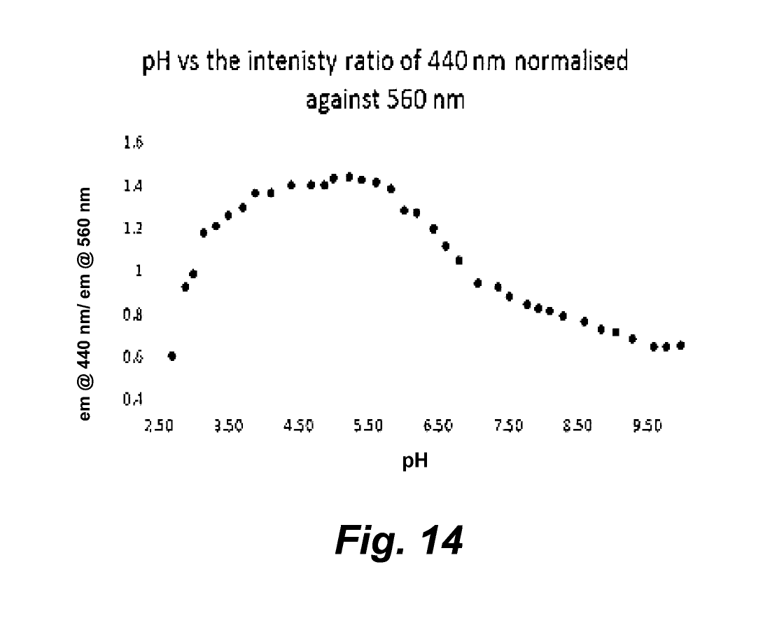

FIG. 14 shows the change in fluorescent intensity for PhenCyano Rhenium molecular probe expressed as a ratio of intensity of emission at 440 nm/560 nm over the pH range of 2.5-10.

Analysis of the fluorescence emission of the probe using fluorescence emission spectroscopy recorded on a Varian CaryEclipse Fluoriometer shows the probe to be pH sensitive in the blue region whilst being insensitive in the red region. The fluorescence emission changes in the blue region were analysed with respect to the insensitive red region and plotted as a function of pH to give a ratiometric analysis curve.

The ratio plot was used to estimate the pH in acidic compartments when incubated with the probe. For example, the ratio of emission at 440 nm to 560 nm in an acidic compartment as measured by confocal microscopy was found to be 1.29. If this ratio is then applied to the graph shown in FIG. 14 and it is assumed that the pH of this compartment is pH<6, then the pH of the acidic compartment was measured to be ˜pH 3.70. This is consistent with literature reports on the pH of the lysosome.

The data demonstrates that the PhenCyano rhenium molecular probe functions as a pH sensor for acidic compartments.

### Example 15—Increased Binding of Rhenium Probes to Cancer Cells

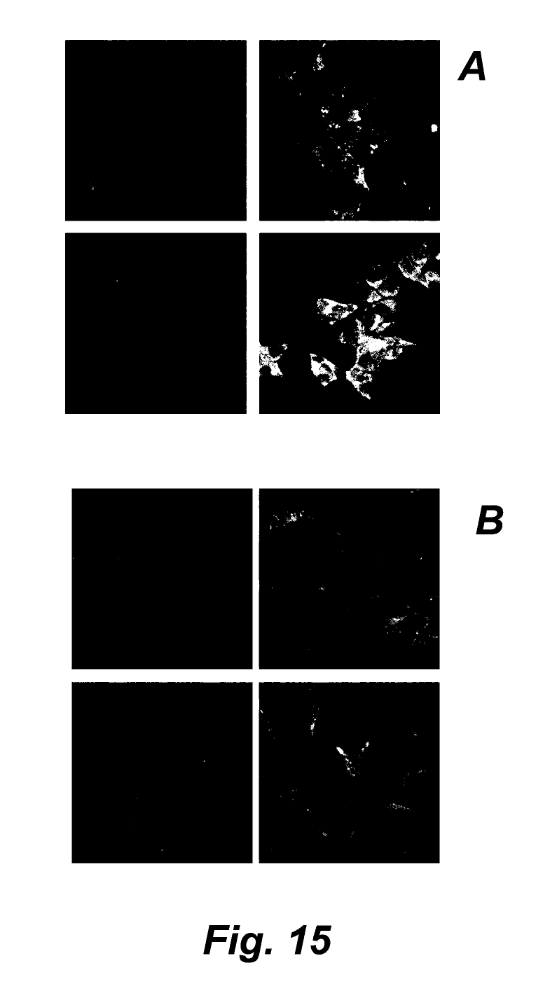

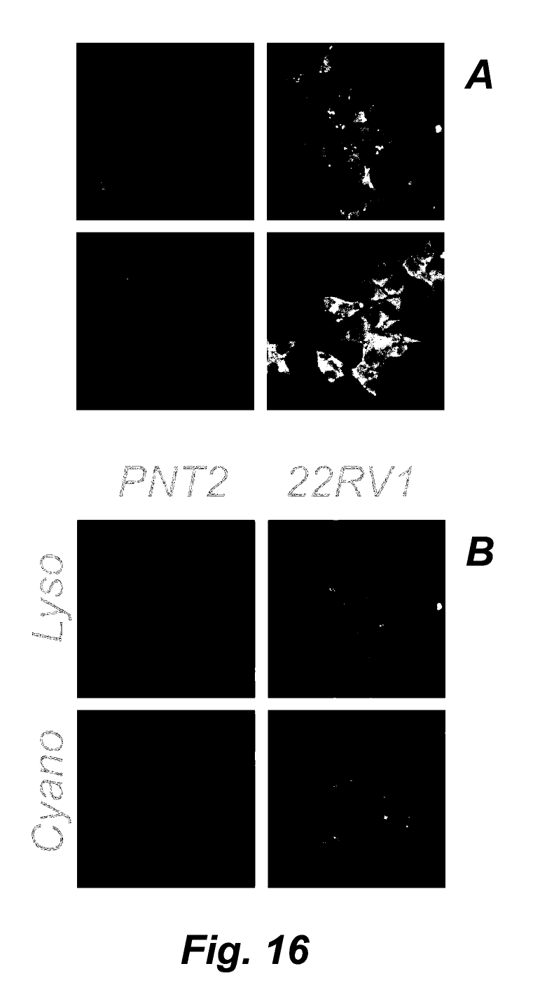

The staining of malignant and non-malignant cells with the PhenCyano and PhenPyridyl probes was investigated, using prostate cancer cells as representative cells. The results are shown in FIGS. 15 and 16.

FIG. 15 shows the labelling of the PhenCyano (“Cyano”) probe and the PhenPyridyl (“Lyso”) probe in prostate cells. The cells labelled PNT2 and PNT1a are non-malignant and the 22RV1 and LNCaP cells are malignant. Panel A shows comparison of PNT2 and 22RV1 cells with the probes. Panel B shows comparison of PNT1a and LNCaP cells with the probes. In this figure, the imaging was adjusted so that all were captured at the same level with identical settings.

FIG. 16 shows the same image as provided in FIG. 15 Panel A at different levels. FIG. 16A shows imaging captured at the same level with identical settings. FIG. 16B show imaging at an optimal level.

The results demonstrate that independent of imaging of the cells, the labelling of cancer cells is increased over non-malignant cells and potentially permits cancerous cells to be distinguished from non-cancerous cells using the probes, such as for diagnostic or prognostic purposes.

### Example 16—Kits for Cell Imaging

An example of a kit using the complexes comprising a transition metal carbonyl compound, a conjugated bidentate ligand and a tetrazolato compound for imaging of lipids in cells is described. Reference is made here to use of the PhenCyano and PhenPyridyl probes, however it will be appreciated that other probes are suitable.

1. Kit Components

(i) PhenCyano probe (tricarbonyl-(4-cyanophenyltretrazolato)-phenanthroline-rhenium) and/or PhenPyridyl probe (tricarbonyl-(3-pyridyltretrazolato) phenanthrolin rhenium (I)) provided in solid form or dissolved in DMSO (10 mM).

(ii) Dilution medium for addition of probes to cells—typically sterile PBS, sterile water or sterile cell culture medium without serum.

One or more other components, including positive and negative lipid controls.

2. Instructions for Using the Probes for Labelling and Detection of Lipids in Cells

Kits are to be stored at room temperature in a dark place. Working solutions of probes are to be prepared only at the time of use, as the probe has reduced solubility in aqueous solutions. If the probes are provided in solid form, a stock solution is required. To prepare a 10 mM stock solution in DMSO: dissolve 1 mg of probe in 160 μl of DMSO. The 10 mM stock solution in DMSO (supplied or prepared by user) must then be diluted to a working solution concentration. Preparation of a typical working solution would require a 1/1000 or 1/500 dilution of the 10 mM stock solution in DMSO into PBS solution.

For tissues, the tissues are isolated in PBS (or other physiological media) and mounted on a coverslip. For cells, these are grown as per normal practice on coverslips.

The media is to be removed and replaced with 10-20 μM solution of molecular probes in PBS (or appropriate physiological media/cell media) at a dilution of stock solution from 1/1000-1/500, for 15-30 minutes at physiologically appropriate temperature (37° C. for cell culture or 25° C. for insect larvae).

Samples are washed for one minute in PBS. If co-staining is performed, the samples are washed in PBS for 30 seconds, before incubating with counterstain.

Tissues are mounted in optical coupling gel, to prevent dehydration prior to imaging and to maintain tissue integrity. For cells, the coverslip is mounted in PBS for immediate imaging.

Samples are to be imaged immediately following staining.

Following fixation (paraffin embedding or alcohol based fixation) samples are washed three times for five minutes in PBS at room temperature.

Samples are incubated with 10-20 μM solution of the molecular probe in PBS (1/1000-1/500 dilution of stock) for 20-30 minutes for alcohol or paraformaldehyde fixed samples, or 40 minutes to one hour for paraffin embedded tissue sections at room temperature, with agitation.

Samples are washed three times for five minutes in PBS at room temperature, with agitation and mounted in 80% glycerol for imaging. Samples can be stored overnight at room temperature in a dark cupboard.

Rhenium molecular probes may be excited by a UV or blue light sources (eg 405 nm). Image collection is performed with a wideband pass filter within the range of 500-650 nm, or narrowband pass filter within this emission range. Photobleaching may occur with mercury light sources if multiple colour imaging is being performed, i.e. if the sample is excited at multiple wavelengths at once.

For two-photon and confocal microscopy, rhenium molecular probes are excited at 800-830 nm using a two-photon pulse laser or 405 steady state laser and detection in the range of 490-670 nm, with an emission maxima at around 570.

Although the present disclosure has been described with reference to particular embodiments, it will be appreciated that the disclosure may be embodied in many other forms. It will also be appreciated that the disclosure described herein is susceptible to variations and modifications other than those specifically described. It is to be understood that the disclosure includes all such variations and modifications. The disclosure also includes all of the steps, features, compositions and compounds referred to, or indicated in this specification, individually or collectively, and any and all combinations of any two or more of the steps or features.

Also, it is to be noted that, as used herein, the singular forms “a”, “an” and “the” include plural aspects unless the context already dictates otherwise.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Reference to any prior art in this specification is not, and should not be taken as, an acknowledgment or any form of suggestion that this prior art forms part of the common general knowledge in any country.

The subject headings used herein are included only for the ease of reference of the reader and should not be used to limit the subject matter found throughout the disclosure or the claims. The subject headings should not be used in construing the scope of the claims or the claim limitations.

The description provided herein is in relation to several embodiments which may share common characteristics and features. It is to be understood that one or more features of one embodiment may be combinable with one or more features of the other embodiments. In addition, a single feature or combination of features of the embodiments may constitute additional embodiments.

All methods described herein can be performed in any suitable order unless indicated otherwise herein or clearly contradicted by context. The use of any and all examples, or exemplary language (e.g., “such as”) provided herein, is intended merely to better illuminate the example embodiments and does not pose a limitation on the scope of the claimed invention unless otherwise claimed. No language in the specification should be construed as indicating any non-claimed element as essential.

Future patent applications may be filed on the basis of the present application, for example by claiming priority from the present application, by claiming a divisional status and/or by claiming a continuation status. It is to be understood that the following claims are provided by way of example only, and are not intended to limit the scope of what may be claimed in any such future application. Nor should the claims be considered to limit the understanding of (or exclude other understandings of) the present disclosure. Features may be added to or omitted from the example claims at a later date.

Although the present disclosure has been described with reference to particular examples, it will be appreciated by those skilled in the art that the disclosure may be embodied in many other forms.

1.-51. (canceled)

52. A method of labelling a lipid, the method comprising exposing the lipid to a complex comprising a transition metal tricarbonyl compound, a conjugated bidentate diimine ligand and a tetrazolato compound, and thereby labelling the lipid by binding the complex to the lipid.

53. The method according to claim 52, wherein the transition metal tricarbonyl compound comprises a transition metal ion comprising Re(I) and/or Ir(III).

54. The method according to claim 52, wherein the lipid comprises one or more of a polar lipid, a non-neutral lipid, a steroid, a sphingomyelin, a sphingosine, a neutral triglyceride, a monosialotetrahexosylganglioside, a phosphatidylethanolamine, a lysophosphatidic acid, a cholesterol, a cholesterol ester, a steroid compound, a steroid hormone, a progesterone and/or a derivative of any of the aforementioned.

55. The method according to claim 52, wherein the conjugated bidentate diimine ligand comprises a phenanthroline compound and/or a bipyridine compound.

56. The method according to claim 55, wherein the phenanthroline compound comprises a 1,10-phenanthroline and/or a substituted derivative thereof, and the bipyridine compound comprises a 2,2′-bipyridine and/or a substituted derivative thereof.

57. The method according to claim 56, wherein the tetrazolato compound comprises a 4-cyanophenyltetrazolate and/or a substituted derivative thereof, a 3-pyridyltetrazolate and/or a substituted derivative thereof, and/or a 4-methyl phenyl carbonate and/or a substituted derivative thereof.

58. The method according to claim 57, wherein the complex has one of the following chemical structures: 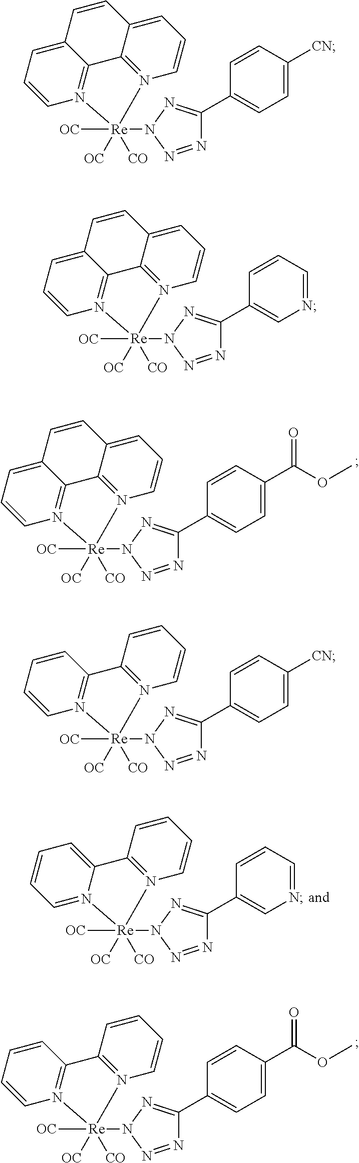
and/or a salt, solvate, tautomer or stereoisomer of any of the aforementioned chemical structures.

59. The method according to claim 52, wherein the method comprises labelling a lipid in a cellular structure selected from one or more of an endosome, a lysosome, an autophagosome a lipid droplet, and endoplasmic reticulum.

60. The method according to claim 52, wherein the method comprises labelling a lipid in a cell selected from one or more of a live cell, an in vitro cell, an in vivo cell, an ex vivo cell, a dead cell, a non-fixed cell, a sorted cell, a stained cell, a cell in a subject, a cell isolated from a subject, a cell for which diagnostic and/or prognostic analysis is to be undertaken, a cancerous cell, a non-cancerous cell, and/or a cell associated with a disease, condition or state associated with altered or dysfunctional lipid intake, metabolism, processing, biogenesis and/or or accumulation.

61. The method according to claim 52, wherein the method comprises labelling a lipid in a biological sample selected from a cell sample, a sample of live cells, a cell extract, a fixed cell, a biopsy, a bodily fluid sample, a blood sample, a urine sample, a saliva sample and/or an extract, component, derivative, processed form or purified form of any of the aforementioned.

62. The method according to claim 52, wherein the method is used to detect a lipid, to detect an endosome, a lysosome, an autophagosome, endoplasmic reticulum and/or a lipid droplet, to visualize an endosome, lysosome, an autophagosome, endoplasmic reticulum and/or a lipid droplet, for intracellular imaging of a live cell, for detecting a disease, condition or state in a subject, to screen for the presence or absence of cancer, for identifying a subject suffering from, or susceptible to, a disease, condition or state, for diagnosis and/or prognosis, to identify a subject suitable for treatment, for identifying a biomarker, to detect an anabolic steroid, to determine the level of an anabolic steroid, and/or for testing for the presence or absence of an anabolic steroid.

63. An intracellular imaging agent, the agent comprising a complex comprising a transition metal tricarbonyl compound, a conjugated bidentate diimine ligand and a tetrazolato compound.

64. The intracellular imaging according to claim 63, wherein the transition metal tricarbonyl compound comprises a transition metal ion comprising Re(I) and/or Ir(III).

65. The intracellular imaging agent according to claim 63, wherein the conjugated bidentate diimine ligand comprises a phenanthroline compound and/or a bipyridine compound.

66. The intracellular imaging agent according to claim 65, wherein the phenanthroline compound comprises a 1,10-phenanthroline and/or a substituted derivative thereof, and the bipyridine compound comprises a 2,2′-bipyridine and/or a substituted derivative thereof.

67. The intracellular imaging agent according to claim 66, wherein the tetrazolato compound comprises a 4-cyanophenyltetrazolate and/or a substituted derivative thereof, a 3-pyridyltetrazolate and/or a substituted derivative thereof, and/or a 4-methyl phenyl carbonate and/or a substituted derivative thereof.

68. The intracellular imaging agent according to claim 67, wherein the complex has one of the following chemical structures: 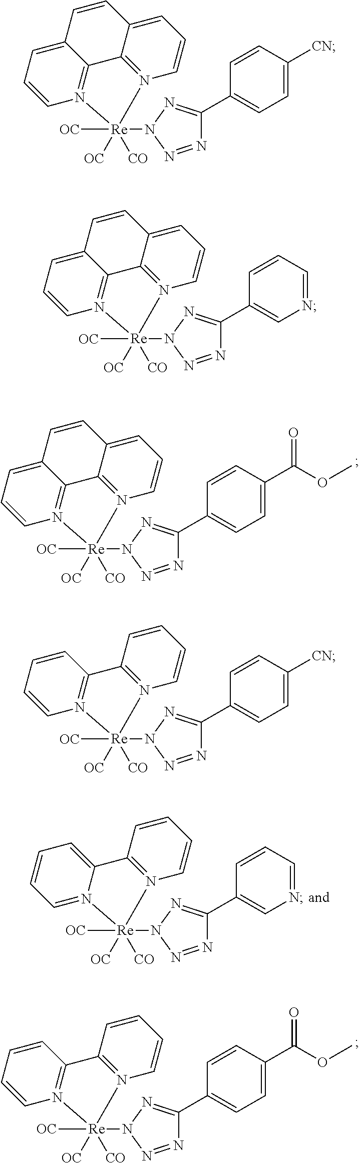
and/or a salt, solvate, tautomer or stereoisomer of any of the aforementioned chemical structures.

69. A kit for intracellular imaging of a cell, the kit comprising an agent according to claim 63.

70. A method of detecting a cancer or a disease, condition or state associated with altered or dysfunctional lipid intake, metabolism, processing, biogenesis or accumulation in a subject, the method comprising exposing one or more cells from the subject to a transition metal tricarbonyl compound, a conjugated bidentate diimine ligand and a tetrazolato compound, and detecting the cancer or the disease, condition or state on the basis of the cells labeled with the agent.

71. The method according to claim 70, wherein the cell is a live cell, an in vitro cell, an in vivo cell, an ex vivo cell, a dead cell, a non-fixed cell, a sorted cell, a stained cell, a cell in a subject, a cell for which diagnostic and/or prognostic analysis is to be undertaken, a cancerous cell, a prostate cancer cell, a non-cancerous cell, and/or a cell associated with a disease, condition or state associated with altered or dysfunctional lipid intake, metabolism, processing, biogenesis and/or or accumulation.

72. A method of identifying a compound for labelling a lipid, the method comprising:
providing a candidate complex comprising a transition metal tricarbonyl compound, a conjugated bidentate diimine ligand and a tetrazolato compound;
determining the ability of the candidate complex to bind to a lipid; and
identifying the candidate complex as a compound for labelling a lipid on the basis of the complex binding to the lipid.
